SEQUENCE LISTING (11 sequences)

SEQ ID NO: 1 - protein, 80 aa, Artificial Sequence
  Synthetic mutant protein
TFKMNTAQKAHYEKFINALENALKTRHIPAGAVIDMLAEINTEALALDYQIVDKKPGTSIAQGTKAAALRKRFIPKKIKA

SEQ ID NO: 2 - protein, 81 aa, Artificial Sequence
  Synthetic mutant protein
FKMNTAQKAHYEAFIKVLEKAAERNPIDAQVVVEALGAVNIDALAKNLNYQVIDKKPGTDIATGTKAAELRKRFVPKKIKA

SEQ ID NO: 3 - protein, 78 aa, Artificial Sequence
  Synthetic mutant protein
FKMNTAQKAHYEAFISGLENAVKDNPMTAQNVKEGLDLANVGAAALNFKIVDKKAGTEIAKGTKAAELRKRFVPKKKA

SEQ ID NO: 4 - protein, 79 aa, Artificial Sequence
  Synthetic mutant protein
FKMNTAQNAHYEAFISGLERGAKDNPMLAQVVKAGLDLANDGAAALNYKIVDKKPGTDIAKGTKAAELRKRFIPKKIKT

SEQ ID NO: 5 - protein, 77 aa, Artificial Sequence
  Synthetic mutant protein
FKMNTAQKAHYEAFIADLERAAKDNPMPAHIVKAGLDAANAIAATLNFKIVDKKAGTEIAKGTKAAELRKRFVPKKK

SEQ ID NO: 6 - protein, 395 aa, Artificial Sequence
  Synthetic chimeric protein
TFKMNTAQKAHYEKFINALENALKTRHIPAGAVIDMLAEINTEALALDYQIVDKKPGTSIAQGTKAAALRKRFIPKKIKAFKMNTAQKAHYEAFIKVLEKAAERNPIDAQVVVEALGAVNIDALAKNLNYQVIDKKPGTDIATGTKAAELRKRFVPKKIKAFKMNTAQKAHYEAFISGLENAVKDNPMTAQNVKEGLDLANVGAAALNFKIVDKKAGTEIAKGTKAAELRKRFVPKKKAFKMNTAQNAHYEAFISGLERGAKDNPMLAQVVKAGLDLANDGAAALNYKIVDKKPGTDIAKGTKAAELRKRFIPKKIKTFKMNTAQKAHYEAFIADLERAAKDNPMPAHIVKAGLDAANAIAATLNFKIVDKKAGTEIAKGTKAAELRKRFVPKKK

SEQ ID NO: 7 - protein, 317 aa, Artificial Sequence
  Synthetic chimeric protein
TFKMNTAQKAHYEKFINALENALKTRHIPAGAVIDMLAEINTEALALDYQIVDKKPGTSIAQGTKAAALRKRFIPKKIKAFKMNTAQKAHYEAFIKVLEKAAERNPIDAQVVVEALGAVNIDALAKNLNYQVIDKKPGTDIATGTKAAELRKRFVPKKIKAFKMNTAQKAHYEAFISGLENAVKDNPMTAQNVKEGLDLANVGAAALNFKIVDKKAGTEIAKGTKAAELRKRFVPKKKAFKMNTAQNAHYEAFISGLERGAKDNPMLAQVVKAGLDLANDGAAALNYKIVDKKPGTDIAKGTKAAELRKRFIPKKIK

SEQ ID NO: 8 - DNA, 1185 bp, Artificial Sequence
  Synthetic coding sequence for chimeric protein
accttcaaaatgaataccgcgcagaaggcccattatgagaagttcatcaatgccctggagaacgccctgaaaacccgccatatccctgctggtgccgttatcgacatgctggccgagattaacaccgaggccctggcactggactatcagatcgtggataaaaaaccgggcaccagcattgcacagggtaccaaggccgccgcactgcgtaaacgttttattcctaagaaaattaaagcattcaagatgaataccgcacagaaagcacattacgaagcattcattaaagtgctggagaaggccgccgagcgcaacccgattgacgcacaggttgttgttgaagcactgggcgccgttaacatcgacgccctggcaaaaaacctgaactatcaggtgattgacaagaagccgggcaccgatattgccaccggtaccaaggccgcagagctgcgcaagcgcttcgtgccgaagaaaattaaagcctttaaaatgaacaccgcccagaaagcccattacgaggcattcatcagcggtctggaaaatgccgtgaaggataatccgatgaccgcacagaacgttaaagaaggcctggacctggcaaatgtgggcgccgcagccctgaactttaagattgtggataagaaagcaggtaccgagattgccaagggcaccaaagccgcagaactgcgcaaacgctttgtgccgaagaaaaaagcctttaagatgaataccgcccagaacgcccactacgaagcatttattagcggtctggagcgcggtgccaaagataacccgatgctggcacaggttgtgaaggccggcctggatctggccaatgatggtgccgccgcactgaactacaaaattgtggataaaaagccgggcaccgacatcgccaaaggtaccaaagccgccgaactgcgcaaacgtttcattccgaagaaaattaaaaccttcaaaatgaataccgcccaaaaggcacactatgaggcattcattgccgatctggaacgcgccgccaaggacaatcctatgccggcccatattgtgaaagcaggtctggatgccgccaatgcaatcgccgccaccctgaatttcaagatcgtggacaagaaggccggcacagaaatcgccaaaggcaccaaggccgcagaactgcgcaagcgctttgtgccgaaaaagaaa

SEQ ID NO: 9 - DNA, 1185 bp, Artificial Sequence
  Synthetic codon optimized coding sequence for chimeric protein
acatttaagatgaacaccgcccaaaaggcccattacgagaaattcatcaacgccctggaaaacgccctgaagacccgtcatattcctgctggtgccgtgattgatatgctggccgagattaacaccgaagccctggccctggactaccagatcgtggataagaaaccgggcaccagtattgcccaaggtaccaaggccgccgcactgcgcaagcgctttatcccgaaaaagattaaagccttcaagatgaacaccgcccaaaaagcccattacgaggccttcattaaagtgctggaaaaagccgccgagcgtaatccgatcgatgcacaggtggtggttgaggccctgggcgcagtgaatattgacgccctggcaaaaaatctgaactaccaggtgatcgacaaaaagccgggcaccgacattgccaccggtaccaaagcagcagaactgcgcaaacgctttgtgccgaagaaaattaaggccttcaaaatgaacaccgcccagaaagcccattatgaggcattcattagcggcctggagaacgccgttaaagacaacccgatgacagcccagaacgtgaaagaaggtctggacctggccaatgtgggcgcagcagccctgaatttcaaaattgtggataagaaggccggcaccgagattgccaaaggcaccaaggccgccgagctgcgcaagcgcttcgtgccgaagaaaaaagcctttaagatgaataccgcacagaacgcccattacgaagccttcatcagcggtctggaacgtggcgcaaaggataacccgatgctggcccaggttgttaaagccggtctggatctggcaaacgatggcgccgccgcactgaactataaaatcgtggacaagaagccgggtaccgatattgccaagggcaccaaagcagccgaactgcgtaaacgcttcatcccgaagaagattaaaacctttaaaatgaacaccgcacaaaaggcccactacgaagcctttatcgccgatctggaacgtgcagccaaagacaatccgatgccggcccacattgttaaggccggtctggacgcagcaaacgccatcgccgccaccctgaactttaaaatcgtggacaaaaaggccggtaccgaaattgccaagggcaccaaggccgccgagctgcgcaaacgttttgttccgaaaaagaaa

SEQ ID NO: 10 - DNA, 960 bp, Artificial Sequence
  Synthetid coding sequence for chimeric protein
accttcaaaatgaataccgcgcagaaggcccattatgagaagttcatcaatgccctggagaacgccctgaaaacccgccatatccctgctggtgccgttatcgacatgctggccgagattaacaccgaggccctggcactggactatcagatcgtggataaaaaaccgggcaccagcattgcacagggtaccaaggccgccgcactgcgtaaacgttttattcctaagaaaattaaagcattcaagatgaataccgcacagaaagcacattacgaagcattcattaaagtgctggagaaggccgccgagcgcaacccgattgacgcacaggttgttgttgaagcactgggcgccgttaacatcgacgccctggcaaaaaacctgaactatcaggtgattgacaagaagccgggcaccgatattgccaccggtaccaaggccgcagagctgcgcaagcgcttcgtgccgaagaaaattaaagcctttaaaatgaacaccgcccagaaagcccattacgaggcattcatcagcggtctggaaaatgccgtgaaggataatccgatgaccgcacagaacgttaaagaaggcctggacctggcaaatgtgggcgccgcagccctgaactttaagattgtggataagaaagcaggtaccgagattgccaagggcaccaaagccgcagaactgcgcaaacgctttgtgccgaagaaaaaagcctttaagatgaataccgcccagaacgcccactacgaagcatttattagcggtctggagcgcggtgccaaagataacccgatgctggcacaggttgtgaaggccggcctggatctggccaatgatggtgccgccgcactgaactacaaaattgtggataaaaagccgggcaccgacatcgccaaaggtaccaaagccgccgaactgcgcaaacgtttcattccgaagaaaattaaaaccttcaaa

SEQ ID NO: 11 - DNA, 960 bp, Artificial Sequence
  Synthetic codon optimized coding sequence for chimeric protein
acatttaagatgaacaccgcccaaaaggcccattacgagaaattcatcaacgccctggaaaacgccctgaagacccgtcatattcctgctggtgccgtgattgatatgctggccgagattaacaccgaagccctggccctggactaccagatcgtggataagaaaccgggcaccagtattgcccaaggtaccaaggccgccgcactgcgcaagcgctttatcccgaaaaagattaaagccttcaagatgaacaccgcccaaaaagcccattacgaggccttcattaaagtgctggaaaaagccgccgagcgtaatccgatcgatgcacaggtggtggttgaggccctgggcgcagtgaatattgacgccctggcaaaaaatctgaactaccaggtgatcgacaaaaagccgggcaccgacattgccaccggtaccaaagcagcagaactgcgcaaacgctttgtgccgaagaaaattaaggccttcaaaatgaacaccgcccagaaagcccattatgaggcattcattagcggcctggagaacgccgttaaagacaacccgatgacagcccagaacgtgaaagaaggtctggacctggccaatgtgggcgcagcagccctgaatttcaaaattgtggataagaaggccggcaccgagattgccaaaggcaccaaggccgccgagctgcgcaagcgcttcgtgccgaagaaaaaagcctttaagatgaataccgcacagaacgcccattacgaagccttcatcagcggtctggaacgtggcgcaaaggataacccgatgctggcccaggttgttaaagccggtctggatctggcaaacgatggcgccgccgcactgaactataaaatcgtggacaagaagccgggtaccgatattgccaagggcaccaaagcagccgaactgcgtaaacgcttcatcccgaagaagattaaaacctttaaa

(19) United States
(12) Patent Application Publication
(10) Pub. No.: US 2023/0355734 A1
(43) Pub. Date: Nov. 9, 2023

(54) A FACTOR H BINDING PROTEIN B (FHBB) BASED CHIMERIC VACCINE FOR THE PREVENTION AND TREATMENT OF PERIODONTAL DISEASE

(71) Applicant: VIRGINIA COMMONWEALTH UNIVERSITY, Richmond, VA (US)

(72) Inventor: Richard T. MARCONI, Modlothian, VA (US)

(21) Appl. No.: 18/030,160

(22) PCT Filed: Oct. 5, 2021
(86) PCT No.: PCT/US2021/053511

§ 371 Date: Apr. 4, 2023

Related U.S. Application Data
(60) Provisional application No. 63/087,389, filed on Oct. 5, 2020.

(51) Int. Cl. A61K 39/02; A61K 39/40; A61P 37/04

(52) U.S. Cl. CPC A61K 39/0225; A61K 39/40; A61P 37/04

(57) ABSTRACT

Provided herein are recombinant Factor H Binding Protein B (FhbB) chimeric proteins comprising several different mutant variants of the Treponema denticola Factor H binding protein B (FhbB). The mutant variants cannot bind Factor H. The chimeric proteins are used to vaccinate subjects against periodontal disease either systemically and/or by direct application of antibodies generated against the chimeric proteins to the oral cavity (e.g. the gums) of a patient to prevent and/or treat periodontal disease.

### CROSS-REFERENCE TO RELATED APPLICATIONS

This application claims benefit of U.S. Provisional patent application 63/087,389 filed Oct. 5, 2021.

### SEQUENCE LISTING

This application includes as the Sequence Listing the complete contents of the accompanying text file “Sequence.txt”, created Oct. 5, 2021, containing 16.4 kilobytes, hereby incorporated by reference.

### BACKGROUND OF THE INVENTION

### Field of the Invention

The invention generally relates to chimeric proteins comprising mutated forms of several different variants of the Treponema denticola Factor H Binding Protein B (FhbB). In particular, the chimeric proteins are used as a vaccine, or to generate antibodies, for treating and/or preventing periodontal disease.

### Description of Related Art

Periodontal disease (PD) refers to a broad range of inflammatory conditions of the gingiva and periodontium. The socioeconomic costs of PD are staggering and impact the global health economy. Treatment for PD is expensive, invasive and unavailable to a vast majority of the global population. PD is a risk factor for several systemic disorders including cardiovascular disease, rheumatoid arthritis, adverse birth outcomes, and Alzheimer's disease (Chapple & Genco, 2013; Li, Kolltveit, Tronstad, & Olsen, 2000; Schulz, Schlitt, Hofmann, Schaller, & Reichert, 2020). The microbial etiology of PD is complex with several hundred bacterial species inhabiting the subgingival crevice (Donos, 2018; Kinane, Stathopoulou, & Papapanou, 2017). As PD develops, a transition in the microbiome of the subgingival crevice from predominately Gram-positive to Gram-negative bacteria and spirochetes of the genus Treponema occurs (Socransky & Haffajee, 2005). Of the 70 species and phylotypes of oral treponemes that have been identified in the human oral cavity (Paster et al., 2001; Paster et al., 1991), the most abundant in periodontal pockets is Treponema denticola (Ellen, 2006). T. denticola is an anaerobe with potent proteolytic capabilities (Ishihara, Miura, Kuramitsu, & Okuda, 1996; Miao, Fenno, Timm, Joo, & Kapila, 2011).

The T. denticola virulence factors, dentilisin (Chi, Qi, & Kuramitsu, 2003; Goetting-Minesky et al., 2012) and Factor H (FH) binding protein B (FhbB) (McDowell et al., 2011; McDowell, Frederick, Stamm, & Marconi, 2007), have been postulated to be key contributors to disease progression. Dentilisin is multi-subunit protease that cleaves a diverse array of substrates including immune regulatory proteins (reviewed in (McDowell, Miller, Mallory, & Marconi, 2012)). FhbB is an approximate 11.4 kDa lipoprotein that binds to the CCP7 domain of human FH and to plasminogen (McDowell et al., 2005; Tegels, Oliver, Miller, & Marconi, 2018). In mammals, FH plays a central role in controlling complement activation via the alternative pathway (Ruddy & Austen, 1969, 1971) by serving as a cofactor in the factor I (FI)-mediated cleavage of C3b. In addition, it inhibits the formation of C3 convertase complex and accelerates decay of preexisting complex (Zipfel & Skerka, 2009). Numerous pathogens, including T. denticola, exploit the negative regulatory activity of FH to evade complement (McDowell et al., 2012). FH bound to FhbB on the cell surface is competent to serve as a cofactor for FI mediated cleavage of the opsonin, C3b (McDowell, Huang, Fenno, & Marconi, 2009). The essential role that the FhbB-FH interaction plays in PD pathogenesis was revealed through the generation and analysis of T. denticola fhbB deletion mutants. While wild-type T. denticola strains are complement resistant, deletion of fhbB renders cells highly sensitive to human serum (McDowell et al., 2009). The outcome of FH and plasminogen binding to T. denticola is unique in that both ligands are ultimately degraded by dentilisin (McDowell et al., 2011; Tegels et al., 2018). It has been hypothesized that as the T. denticola population proliferates with disease progression, the rate of FH cleavage in gingival crevicular fluid may exceed its rate of replenishment resulting in local depletion of FH (McDowell et al., 2012). In the absence of FH, C3b deposition on host tissues would ensue leading to self-attack by the immune system and local immune dysregulation. Tissue degradation would release nutrients and create an expanded anaerobic environment favorable to the periopathogen community in general.

FhbB is unique to T. denticola and universal among strains. Three antigenically distinct FhbB variants referred to as FhbB types 1, 2 and 3 have been identified (Miller et al., 2012; Miller et al., 2013). The structure of FhbB1 has been determined at 1.7 Å resolution (Miller et al., 2012; Miller, McDowell, Bell, & Marconi, 2011) and its FH and plasminogen binding domains identified (Miller et al., 2012; Tegels et al., 2018). While FhbB crystallized as a dimer, it's extensive water interface between monomers and weak dimer dissociation constant (217±40.5 μM) suggest that the biologically active form is the monomer (Miller et al., 2012). Site-directed amino acid substitution analyses of FhbB1 revealed that FH and plasminogen bind to negative and positively charged faces of the FhbB protein, respectively. It has been demonstrated that anti-FhbB1 antibody can block FH binding and thereby prevent its cleavage by dentilisin (Miller et al., 2016).

There is a need for agents that protect against PD. In particular, it would be advantageous to have available an FhbB based vaccinogen that could provide protection against the pathogenesis of Treponema denticola.

### SUMMARY OF THE INVENTION

Other features and advantages of the present invention will be set forth in the description of invention that follows, and in part will be apparent from the description or may be learned by practice of the invention. The invention will be realized and attained by the compositions and methods particularly pointed out in the written description and claims hereof.

There are three major variants of the T. denticola FhbB protein that are referred to as FhbB1, FhbB2 and FhbB3. There are additional minor variants of FhbB3. The FhbB protein plays a critical role in the pathogenesis of T. denticola, a causative agent of periodontal disease. FhbB binds to a protein called Factor H (FH), which all mammals produce. When T. denticola binds FH via the FhbB protein, it results in the degradation of the protein causing dysregulation of the immune system in the subgingival crevice, causing or contributing to periodontal disease.

The invention encompasses a series of proteins that are laboratory designed, recombinant chimeric polypeptides comprising several different, genetically engineered mutants of T. denticola FhbB, and methods of using the chimeric polypeptides to prevent and treat PD. The mutations that are introduced result in forms of the FhbB proteins that no longer bind FH. Administration of a chimeric protein comprising a plurality of mutant FhbB proteins to a subject elicits production of antibodies to the chimeras. The production of the antibodies prevents and/or treats PD through at least two distinct but synergistic mechanisms: 1) antibody-mediated complement dependent killing of T. denticola bacteria, which is augmented by 2) antibody-mediated blockage of T. denticola binding to FH, which renders the T. denticola more susceptible to the antibody-mediated complement dependent killing. In additional aspects, antibodies against the chimeras are harvested and administered to a subject in order to prevent and/or treat PD.

It is an object of this invention to provide a recombinant chimeric protein comprising at least one genetically engineered mutant Treponema denticola Factor H Binding Protein B (FhbB) which comprises at least one mutation compared to a wild type FhbB primary sequence, wherein the at least one mutation prevents binding of the at least one genetically engineered mutant T. denticola FhbB to Factor H (FH). In some aspects, the at least one mutation includes a substitution at amino acid position 42, 43, 45, 57, 58, 64, 64, 68, 93 and/or 96 of wild type FhbB primary sequence. In further aspects, the at least one mutation is at one or both of amino acid positions 45 and 58. In additional aspects, the at least one mutation is an alanine substitution. In other aspects, the recombinant chimeric protein comprises a plurality of genetically engineered mutant T. denticola FhbBs. In certain aspects, the recombinant chimeric protein comprises 2, 3, 4, 5 or 6 genetically engineered mutant T. denticola FhbBs. In further aspects, the at least one genetically engineered mutant T. denticola FhbB has an amino acid sequence as set forth in SEQ ID NO: 1, SEQ ID NO: 2, SEQ ID NO: 3, SEQ ID NO: 4 or SEQ ID NO: 5. In additional aspects, the recombinant chimeric protein has an amino acid sequence as set forth in: SEQ ID NO: 6 or SEQ ID NO: 7.

Also provided is a vaccine composition, comprising a recombinant chimeric protein comprising at least one genetically engineered mutant Treponema denticola Factor H Binding Protein B (FhbB) which comprises at least one mutation compared to a wild type FhbB primary sequence, wherein the at least one mutation prevents binding of the at least one genetically engineered mutant T. denticola FhbB to Factor H (FH). In some aspects, the at least one mutation includes a substitution at amino acid position 42, 43, 45, 57, 58, 64, 64, 68, 93 and/or 96 of wild type FhbB primary sequence. In further aspects, the at least one mutation is at one or both of amino acid positions 45 and 58. In additional aspects, the at least one mutation is an alanine substitution. In other aspects, the recombinant chimeric protein comprises a plurality of genetically engineered mutant T. denticola FhbBs. In certain aspects, the recombinant chimeric protein comprises 2, 3, 4, 5 or 6 genetically engineered mutant T. denticola FhbBs. In further aspects, the at least one genetically engineered mutant T. denticola FhbB has an amino acid sequence as set forth in SEQ ID NO: 1, SEQ ID NO: 2, SEQ ID NO: 3, SEQ ID NO: 4 or SEQ ID NO: 5. In additional aspects, the recombinant chimeric protein has an amino acid sequence as set forth in: SEQ ID NO: 6 or SEQ ID NO: 7.

The invention also provides a method of preventing and/or treating periodontal disease in a subject in need thereof, comprising, administering to the subject
i) a therapeutically effective amount of a recombinant chimeric protein comprising at least one genetically engineered mutant Treponema denticola Factor H Binding Protein B (FhbB) which comprises at least one mutation compared to a wild type FhbB primary sequence, wherein the at least one mutation prevents binding of the at least one genetically engineered mutant T. denticola FhbB to Factor H (FH). In some aspects, the at least one mutation includes a substitution at amino acid position 42, 43, 45, 57, 58, 64, 64, 68, 93 and/or 96 of wild type FhbB primary sequence. In further aspects, the at least one mutation is at one or both of amino acid positions 45 and 58. In additional aspects, the at least one mutation is an alanine substitution. In other aspects, the recombinant chimeric protein comprises a plurality of genetically engineered mutant T. denticola FhbBs. In certain aspects, the recombinant chimeric protein comprises 2, 3, 4, 5 or 6 genetically engineered mutant T. denticola FhbBs. In further aspects, the at least one genetically engineered mutant T. denticola FhbB has an amino acid sequence as set forth in SEQ ID NO: 1, SEQ ID NO: 2, SEQ ID NO: 3, SEQ ID NO: 4 or SEQ ID NO: 5. In additional aspects, the recombinant chimeric protein has an amino acid sequence as set forth in: SEQ ID NO: 6 or SEQ ID NO: 7; and/or
ii) a therapeutically effective amount of antibodies against a recombinant chimeric protein comprising at least one genetically engineered mutant Treponema denticola Factor H Binding Protein B (FhbB) which comprises at least one mutation compared to a wild type FhbB primary sequence, wherein the at least one mutation prevents binding of the at least one genetically engineered mutant T. denticola FhbB to Factor H (FH). In some aspects, the at least one mutation includes a substitution at amino acid position 42, 43, 45, 57, 58, 64, 64, 68, 93 and/or 96 of wild type FhbB primary sequence. In further aspects, the at least one mutation is at one or both of amino acid positions 45 and 58. In additional aspects, the at least one mutation is an alanine substitution. In other aspects, the recombinant chimeric protein comprises a plurality of genetically engineered mutant T. denticola FhbBs. In certain aspects, the recombinant chimeric protein comprises 2, 3, 4, 5 or 6 genetically engineered mutant T. denticola FhbBs. In further aspects, the at least one genetically engineered mutant T. denticola FhbB has an amino acid sequence as set forth in SEQ ID NO: 1, SEQ ID NO: 2, SEQ ID NO: 3, SEQ ID NO: 4 or SEQ ID NO: 5. In additional aspects, the recombinant chimeric protein has an amino acid sequence as set forth in: SEQ ID NO: 6 or SEQ ID NO: 7. In some aspects, the therapeutically effective amount of the recombinant chimeric protein is administered systemically. In further aspects, the antibodies are monoclonal antibodies. In additional aspects, the therapeutically effective amount of antibodies is administered locally. In yet additional aspects, the therapeutically effective amount of antibodies is administered locally using a sustained-release formulation.

The invention also provides a method of eliciting an immune response to Treponema denticola Factor H Binding Protein B (FhbB) protein in a subject, comprising administering to the subject an amount of a recombinant chimeric protein comprising at least one genetically engineered mutant Treponema denticola Factor H Binding Protein B (FhbB) which comprises at least one mutation compared to a wild type FhbB primary sequence, wherein the at least one mutation prevents binding of the at least one genetically engineered mutant T. denticola FhbB to Factor H (FH). In some aspects, the at least one mutation includes a substitution at amino acid position 42, 43, 45, 57, 58, 64, 64, 68, 93 and/or 96 of wild type FhbB primary sequence. In further aspects, the at least one mutation is at one or both of amino acid positions 45 and 58. In additional aspects, the at least one mutation is an alanine substitution. In other aspects, the recombinant chimeric protein comprises a plurality of genetically engineered mutant T. denticola FhbBs. In certain aspects, the recombinant chimeric protein comprises 2, 3, 4, 5 or 6 genetically engineered mutant T. denticola FhbBs. In further aspects, the at least one genetically engineered mutant T. denticola FhbB has an amino acid sequence as set forth in SEQ ID NO: 1, SEQ ID NO: 2, SEQ ID NO: 3, SEQ ID NO: 4 or SEQ ID NO: 5. In additional aspects, the recombinant chimeric protein has an amino acid sequence as set forth in: SEQ ID NO: 6 or SEQ ID NO: 7, wherein the amount is sufficient to elicit an immune response in the subject. In some aspects, the immune response results in a reduction in the population of T. denticola in the subject. In further aspects, the immune response includes the production of antibodies. In additional aspects, the method comprises a step of harvesting the antibodies from the subject.

The invention also provides a method of producing monoclonal antibodies to the chimeric protein of a recombinant chimeric protein comprising at least one genetically engineered mutant Treponema denticola Factor H Binding Protein B (FhbB) which comprises at least one mutation compared to a wild type FhbB primary sequence, wherein the at least one mutation prevents binding of the at least one genetically engineered mutant T. denticola FhbB to Factor H (FH). In some aspects, the at least one mutation includes a substitution at amino acid position 42, 43, 45, 57, 58, 64, 64, 68, 93 and/or 96 of wild type FhbB primary sequence. In further aspects, the at least one mutation is at one or both of amino acid positions 45 and 58. In additional aspects, the at least one mutation is an alanine substitution. In other aspects, the recombinant chimeric protein comprises a plurality of genetically engineered mutant T. denticola FhbBs. In certain aspects, the recombinant chimeric protein comprises 2, 3, 4, 5 or 6 genetically engineered mutant T. denticola FhbBs. In further aspects, the at least one genetically engineered mutant T. denticola FhbB has an amino acid sequence as set forth in SEQ ID NO: 1, SEQ ID NO: 2, SEQ ID NO: 3, SEQ ID NO: 4 or SEQ ID NO: 5. In additional aspects, the recombinant chimeric protein has an amino acid sequence as set forth in: SEQ ID NO: 6 or SEQ ID NO: 7, comprising injecting the chimeric protein into a host animal; obtaining spleen cells from the host animal; fusing the spleen cells with myeloma cells to form hybridoma cells; and culturing the hybridoma cells under conditions that permit lymphocytes within the hybridoma cells to produce the monoclonal antibodies. A monoclonal antibody produced by this method is also encompassed.

### BRIEF DESCRIPTION OF THE DRAWINGS

FIG. 1. Generation of recombinant FhbB proteins and chimerics and analysis of FH binding. Panel A depicts a ribbon model structure for FhbB1 with residues previously demonstrated to be required for FH binding highlighted (Miller et al., 2012). Panel B depicts the strategy for the generation of the FhbB chimeric proteins, FhbB-ch4 and FhbB-ch5. Panel C presents the results of ELISA based-FH binding assays. Statistically significant differences in FH binding of the mutated proteins relative to the corresponding wild-type protein are indicated by ** (P<0.001). Recombinant B. burgdorferi VlsE served as the negative control for FH binding.

FIG. 2. FhbB chimerics elicit antibodies that recognize each FhbB type represented in each chimeric. Recombinant proteins (indicated along the x-axis) were screened with anti-FhbB-ch4, anti-FhbB-ch5 antisera or preimmune sera (as indicated) exactly as detailed in the text. Statistically significant differences are indicated * (P<0.0001).

FIG. 3. Anti-FhbB-ch4 antisera recognizes native forms of FhbB and causes cell lysis and cell aggregation. Panel A displays the results of IFA analyses in which strains (as indicated) producing different FhbB type proteins were screened with anti-FhbB-ch4 antisera. The corresponding dark-field images are shown. Panel B displays the results of bactericidal/cell aggregation assays in which cells were incubated with or without anti-FhbB-ch4 antisera in the presence of complement preserved GPS (90 minutes). All methods were as described in the text.

FIG. 4. Anti-FhbB-ch4 antisera blocks FH cleavage by the dentilisin positive strain 35405. Actively growing cells were preincubated with increasing concentrations of anti-FhbB-ch4 antisera and then purified human FH was added (0 or 60 min). The samples were subjected to SDS-PAGE, immunoblotted and screened with anti-FH antisera. The dentilisin deficient strain, SP50, served as a negative control for FH cleavage. The images were cropped for presentation purposes.

### DETAILED DESCRIPTION

Disclosed herein are recombinant, genetically engineered chimeric proteins that comprise mutant forms of one or more FhbB proteins from various strains and/or variants of T. denticola bacteria. To produce the chimeras, native (wildtype) FhbB proteins were genetically modified by introducing mutations at specific positions, in the amino acid sequences of the proteins, that render the resulting mutants incapable of binding FH (for example, residues E45 and D58, which project outward from the negatively charged FH binding interface). When administered as a vaccine, the chimeric FhbB proteins do not bind FH and therefore do not degrade FH. However, administration of the recombinant proteins triggers in vivo production of antibodies that target and bind to diverse strains of T. denticola via the FhbB protein. When antibodies elicited by vaccination bind to T. denticola, the bacterium is killed outright and in fact is rendered more susceptible to killing by antibodies, since FhbB is less stable when not bound to FH. Further, the binding of antibodies to the bacterium prevents T. denticola from degrading FH and thereby aids in the maintenance of a healthy immunological environment. In another aspect, antibodies generated using the proteins can also be used therapeutically, i.e. for antibody therapy. For example, antibodies to the proteins can be applied directly to a site that is or is likely to be affected by periodontal disease caused or exacerbated by T. denticola, thereby preventing and/or treating periodontal disease.

### Definitions

Treponema denticola refers to a Gram-negative, motile, obligate anaerobic, and highly proteolytic spirochete bacterium. T. denticola dwells in a complex and diverse microbial community within the oral cavity, is highly specialized to survive in this environment and is associated with the incidence and severity of human periodontal disease. T. denticola is one of three bacteria that form the Red Complex, the other two being Porphyromonas gingivalis and Tannerella forsythia. Together they form the major virulent pathogens that cause chronic periodontitis. Having elevated T. denticola levels in the mouth is considered one of the main etiological agents of periodontitis.

Factor H is a member of the regulators of complement activation family and is a complement control protein. It is a large (155 kilodaltons), soluble glycoprotein that circulates in human plasma (at typical concentrations of 200-300 micrograms per milliliter). Its principal function is to regulate the alternative pathway of the complement system, ensuring that the complement system is directed towards pathogens or other dangerous material and does not damage host tissue.

A “vaccine composition” as used herein refers to a pharmaceutical composition comprising one or more proteins, polypeptides or peptides comprising antigenic regions to which an immune response is generated when administered to a host. Such compositions may also be referred to herein as “immunogenic compositions”.

“Epitope” (antigenic determinant) refers to the part of an antigen molecule to which an antibody attaches itself.

### The Chimeric Proteins

The chimeric proteins disclosed herein comprise at least one, and generally at least two FhbB proteins from different T. denticola strains, or variants of strains, which have been mutated using genetic engineering technology. The mutations that are introduced prevent the proteins from binding to FH. In some aspects, 2, 3, 4, 5, 7 or 8 or more different FhbB proteins from different T. denticola strains, or variants of strains, are used in each chimera. In preferred embodiments, 4 or 5 FhbB proteins from different T. denticola strains are used in a single chimera.

The wild-type version(s) of any T. denticola FhbB protein may be used in the practice of the invention. Exemplary T. denticola FhbB proteins which are mutated and used in the chimeras disclosed herein include but are not limited to: FhbB1, FhbB2, FhbB3, FhbB3-64, FhbB3-35404, FhbB3-33521 and FhbB3-46.

The wild-type version(s) of a T. denticola FhbB protein from any strain or variant thereof may be used in the practice of the invention. Exemplary strains and variants of strains from which the wild-type proteins are originally found or isolated (i.e. from which the mutants are derived) include but are not limited to: 35405 (e.g. for FhbB1), SP50 (e.g. for FhbB2), 33521 (e.g. for FhbB3), 35404 (e.g. for FhbB3), and SP64 (e.g. for FhbB3).

The mutation(s) that are introduced into the wild-type sequences include any mutation that prevents or at least decreases (e.g. by at least about 50%, preferably at least by 60, 65, 70, 75, 80, 85, 90, 95 or even 100%) the ability of the protein to bind to FH. In some aspects, the mutation(s) that are introduced into the wild-type sequences are alanine (A) substitutions. However, other substitutions may used, for example, one or more substitutions by any common amino acid e.g. by alanine (ala, A), arginine (arg, R) asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y) or valine (val, V); or by various less common or synthetic amino acids, examples of which include but are not limited to: ornithine, hydroxylysine, hydroxyproline, thyroxine, 7-carboxyglutamic acid, selenocysteine, etc. The position(s) may be substituted by any amino acid, as long as the resulting mutant protein does not bind FH or binds FH at a level that is suitable for use in the practice of the invention, e.g. at most about 50% of the level of binding of the native protein In exemplary aspects, the amino acids are substituted by alanine.

In some aspects, mutations are introduced at one or more (at least one) of exemplary positions 42, 43, 45, 57, 64, 64, 68, 93 or 96 of the protein. In exemplary aspects, the mutations are at one or both of E45 and D58. In yet further exemplary aspects, the mutations include one or both of E45A and D58A.

Exemplary mutant FhbB protein amino acid sequences are shown below, together with an indication of the change that is made compared to the wild-type protein (the amino acids that are in bold), and the strain/variant from which the protein originated (in subscript). Signal peptides were not included in the constructs, but they may be included as optional features of the other chimeric constructs. The sequence numbering used herein is based on full-length wild type sequences which include the signal peptide. Thus, for example, SEQ ID NO: 1 shows the E45A (substitution of A for E at position 45 of the full-length protein which includes the signal peptide) whereas without the signal peptide the mutation is at position 22 of the mutant.

FhbB1₃₅₄₀₅
(SEQ ID NO: 1)
TFKMNTAQKAHYEKFINALENALKTRHIPAGAVIDMLAEINTEALALDYQ
IVDKKPGTSIAQGTKAAALRKRFIPKKIKA
FhbB2_{SP50}
(SEQ ID NO: 2)
FKMNTAQKAHYEAFIKVLEKAAERNPIDAQVVVEALGAVNIDALAKNLNY
QVIDKKPGTDIATGTKAAELRKRFVPKKIKA
FhbB3₃₃₅₂₁
(SEQ ID NO: 3)
FKMNTAQKAHYEAFISGLENAVKDNPMTAQNVKEGLDLANVGAAALNFKI
VDKKAGTEIAKGTKAAELRKRFVPKKKA
FhbB3₃₅₄₀₄
(SEQ ID NO: 4)
FKMNTAQNAHYEAFISGLERGAKDNPMLAQVVKAGLDLANDGAAALNYKI
VDKKPGTDIAKGTKAAELRKRFIPKKIKT
FhbB3_{SP64}
(SEQ ID NO: 5)
FKMNTAQKAHYEAFIADLERAAKDNPMPAHIVKAGLDAANAIAATLNFKI
VDKKAGTEIAKGTKAAELRKRFVPKKK

The plurality of mutant FhbB protein sequences that are included in a chimeric protein of the invention may be arranged in any order in the linear primary sequence of a chimera. For example, if a chimeric protein comprises 5 different mutant FhbB proteins, indicated as 1, 2, 3, 4, and 5, the order within the chimera may be 1, 2, 3, 4, 5; or 2, 3, 4, 5, 1; or 3, 4, 5, 1, 2; or 4, 5, 1, 2, 3; 5, 1, 2, 3, 4; or a completely random order such as 1, 3, 5, 2, 4; or 5, 2, 3, 1, 4; etc. Any ordered combination of the 5 sequences in encompassed herein. If multiple identical copies of a mutant are present, the copies may or may not be positioned one after another (in tandem) in the primary sequence i.e. if they are not in tandem, they may be interspersed between other, non-identical mutant sequences. All such variations in the order of the mutants in a chimera are encompassed herein.

The chimeric proteins may or may not contain other elements. For example, linkers (spacers) may be included, i.e. short (such as about 10 amino acids or less) amino acids that are placed between two mutant protein sequences and/or before the first mutant protein sequence or after the last protein sequence of the chimera. Examples of suitable linking sequences include but are not limited to: Gly-Gly-Gly-Ser repeated n times, where n is 1, 2, 3 or 4, short peptide linkers (e.g., 5 or 10 amino acids) and those taught in published US patent applications 20210277414 and 20180369334, the entire contents of each of which is hereby incorporated by reference in entirety.

In other aspects, some amino acid sequences may occur, especially at the carboxy and/or amino terminus of a chimera, that are adventitiously derived from vectors used in the cloning procedure, i.e. they are encoded by nucleic acid sequences which are part of a coding vector and are translated along with the nucleic acid sequence that encodes the mutant protein.

In addition, the present disclosure encompasses modified variants of the polypeptide sequences disclosed herein, as long as the modified variant does not bind or cleave FH but does elicit antibodies to at least one T. denticola FhbB protein, and the antibodies kill T. denticola and/or preventing binding of at least one T. denticola FhbB protein to FH. For example, one or more amino acids in a sequence may be conservatively or non-conservatively substituted by a different natural or non-natural amino acid, or may be modified e.g. by carboxylation, amidation, sulfation, etc. As to amino acid sequences, one of skill will recognize that individual substitutions, deletions or additions to a peptide, polypeptide, or protein sequence which alters, adds or deletes a single amino acid or a small percentage of amino acids in the encoded sequence is a “conservatively modified variant”, as long as the desired activity/activities of the resulting mutant is preserved. In some aspects, the alteration is a substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following groups each contain amino acids that are conservative substitutions for one another: 1) Non-polar—Alanine (A), Leucine (L), Isoleucine (I), Valine (V), Glycine (G), Methionine (M); 2) Aliphatic—Alanine (A), Leucine (L), Isoleucine (I), Valine (V); 3) Acidic—Aspartic acid (D), Glutamic acid (E); 4) Polar—Asparagine (N), Glutamine (Q); Serine (S), Threonine (T); 5) Basic—Arginine (R), Lysine (K); 7) Aromatic—Phenylalanine (F), Tyrosine (Y), Tryptophan (W), Histidine (H); 8) Other—Cysteine (C) and Proline (P).

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, 7-carboxyglutamate, and O-phosphoserine. In embodiments, the amino acid side chain may be a non-natural amino acid side chain.

The term “non-natural amino acid side chain” refers to the functional substituent of compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium, allylalanine, 2-aminoisobutryric acid. Non-natural amino acids are non-proteinogenic amino acids that occur naturally or are chemically synthesized. Such analogs have modified R groups (e.g., norleucine) or modified peptide backbones, but retain the same basic chemical structure as a naturally occurring amino acid. Non-limiting examples include exo-cis-3-Aminobicyclo[2.2.1]hept-5-ene-2-carboxylic acid hydrochloride, cis-2-Aminocycloheptanecarboxylic acid hydrochloride,cis-6-Amino-3-cyclohexene-1-carboxylic acid hydrochloride, cis-2-Amino-2-methylcyclohexanecarboxylic acid hydrochloride, cis-2-Amino-2-methylcyclopentanecarboxylic acid hydrochloride, 2-(Boc-aminomethyl)benzoic acid, 2-(Boc-amino)octanedioic acid, Boc-4,5-dehydro-Leu-OH (dicyclohexylammonium), Boc-4-(Fmoc-amino)-L-phenylalanine, Boc-.beta.-Homopyr-OH, Boc-(2-indanyl)-Gly-OH, 4-Boc-3-morpholineacetic acid, 4-Boc-3-morpholineacetic acid, Boc-pentafluoro-D-phenylalanine, Boc-pentafluoro-L-phenylalanine, Boc-Phe(2-Br)—OH, Boc-Phe(4-Br)—OH, Boc-D-Phe(4-Br)—OH, Boc-D-Phe(3-C1)-OH, Boc-Phe(4-NH.sub.2)-OH, Boc-Phe(3-NO.sub.2)-OH, Boc-Phe(3,5-F2)-OH, 2-(4-Boc-piperazino)-2-(3,4-dimethoxyphenyl)acetic acid purum, 2-(4-Boc-piperazino)-2-(2-fluorophenyl)acetic acid purum, 2-(4-Boc-piperazino)-2-(3-fluorophenyl)acetic acid purum, 2-(4-Boc-piperazino)-2-(4-fluorophenyl)acetic acid purum, 2-(4-Boc-piperazino)-2-(4-methoxyphenyl)acetic acid purum, 2-(4-Boc-piperazino)-2-phenylacetic acid purum, 2-(4-Boc-piperazino)-2-(3-pyridyl)acetic acid purum, 2-(4-Boc-piperazino)-2-[4-(trifluoromethyl)phenyl]acetic acid purum, Boc-.beta.-(2-quinolyl)-Ala-OH, N—Boc-1,2,3,6-tetrahydro-2-pyridinecarboxylic acid, Boc-.beta.-(4-thiazolyl)-Ala-OH, Boc-p-(2-thienyl)-D-Ala-OH, Fmoc-N-(4-Boc-aminobutyl)-Gly-OH, Fmoc-N-(2-Boc-aminoethyl)-Gly-OH, Fmoc-N-(2,4-dimethoxybenzyl)-Gly-OH, Fmoc-(2-indanyl)-Gly-OH, Fmoc-pentafluoro-L-phenylalanine, Fmoc-Pen(Trt)-OH, Fmoc-Phe(2-Br)—OH, Fmoc-Phe(4-Br)—OH, Fmoc-Phe(3,5-F2)-OH, Fmoc-β-(4-thiazolyl)-Ala-OH, Fmoc-β-(2-thienyl)-Ala-OH, 4-(Hydroxymethyl)-D-phenylalanine.

Also encompassed are chimeric proteins comprising one or more affinity tags that facilitate isolation of the protein, e.g. various small peptide sequences (such as Bluetongue virus tag (B-tag), FLAG epitope, Glu-Glu (EE-tag), histidine affinity tag (HAT), HSV epitope, KT3 epitope, Myc epitope, PDZ ligand, Polyarginine (Arg-tag), Polyaspartate (Asp-tag), Polycysteine (Cys-tag), Polyhistidine (His-tag), Polyphenylalanine (Phe-tag), Protein C, S1-tag, S-tag, Streptavadin-binding peptide (SBP), Strep-tag, Small Ubiquitin-like Modifier (SUMO), Ubiquitin, Universal i.e. HTTPHH, VSV-G, etc.); and the like; or longer amino acid sequences such as Albumin-binding protein (ABP), Alkaline Phosphatase (AP), Biotin-carboxy carrier protein (BCCP), Calmodulin binding peptide (CBP), Chloramphenicol Acetyl Transferase (CAT), Cellulose binding domain (CBP), Choline-binding domain (CBD), Dihydrofolate reductase (DHFR), Galactose-binding protein (GBP), Green fluorescent protein (GFP), Glutathione S-transferase (GST), Human influenza hemagglutinin (HA), HaloTag®, Horseradish Peroxidase (HRP), Ketosteroid isomerase (KSI), LacZ, Luciferase, Maltose-binding protein (MBP), NusA, PDZ domain, Profinity eXact, Streptavadin-binding peptide (SBP), Staphylococcal protein A (Protein A), Staphylococcal protein G (Protein G), Streptavadin, T7 epitope, Thioredoxin (Trx), TrpE, etc.

The chimeric proteins of the invention may be labeled with a detectable label, e.g. so as to measure or track activity in vitro or in vivo. Such labels include but are not limited to: radioactive amino acids; various fluorescent reagents (e.g. fluorophores including organic dyes such as Alexa dyes, FITC, TRITC, DyLight fluors; biological fluorophores such as green fluorescent protein (GFP), R-phycoerythrin; quantum dots; etc.); and others that are known in the art.

The amino acid sequences of two exemplary chimeric proteins are shown below, where the sequences are annotated to indicate the location of the amino acid substitutions (in bold) and the sequential order of individual mutants is given just before the sequence. The individual mutants are shown by alternate italicized and non-italicized and underlined font.

FhbB-ch5: in sequential order
(FhbB1₃₅₄₀₅-FhbB2_{SP50}-FhbB3₃₃₅₂₁-
FhbB3₃₅₄₀₄-FhbB3_{SP64})
(SEQ ID NO: 6)
TFKMNTAQKAHYEKFINALEN [symbol] LKTRHIPAGAVIDMLAEINTEALALDYQ
IVDKKPGTSIAQGTKAAALRKRFIPKKIKAFKMNTAQKAHYEAFIKVLEK
AAERNPIDAQVVVEALGAVNIDALAKNLNYQVIDKKPGTDIATGTKAAEL
RKRFVPKKIKAFKMNTAQKAHYEAFISGLEN [symbol] VKDNPMTAQNVKEGLDLA
NVGAAALNFKIVDKKAGTEIAKGTKAAELRKRFVPKKKAFKMNTAQNAHY
EAFISGLERGAKDNPMLAQVVKAGLDLANDGAAALNYKIVDKKPGTDIAK
GTKAAELRKRFIPKKIKTFKMNTAQKAHYEAFIADLER [symbol] AKDNPMPAHIV
K [symbol] GLDAANAIAATLNFKIVDKKAGTEIAKGTKAAELRKRFVPKKK
FhbB-ch4: in sequential order
(FhbB1₃₅₄₀₅-FhbB2_{SP50}-
FhbB3₃₃₅₂₁-FhbB3₃₅₄₀₄)
(SEQ ID NO: 7)
TFKMNTAQKAHYEKFINALEN [symbol] LKTRHIPAGAVIDMLAEINTEALALDYQ
IVDKKPGTSIAQGTKAAALRKRFIPKKIKAFKMNTAQKAHYEAFIKVLEK
AAERNPIDAQVVVEALGAVNIDALAKNLNYQVIDKKPGTDIATGTKAAEL
RKRFVPKKIKAFKMNTAQKAHYEAFISGLEN [symbol] VKDNPMTAQNVKEGLDLA
NVGAAALNFKIVDKKAGTEIAKGTKAAELRKRFVPKKKAFKMNTAQNAHY
EAFISGLERGAKDNPMLAQVVKAGLDLANDGAAALNYKIVDKKPGTDIAK
GTKAAELRKRFIPKKIK

Also encompassed herein are nucleic acid sequences that encode the disclosed polypeptides and variants of the polypeptides. Due to the redundancy of the genetic code, several nucleotide sequences can encode a given polypeptide and all such nucleic acid sequences are encompassed herein. Further, the nucleic acids may, for example, be based strictly on a wild-type coding sequence, except for the particular mutants (e.g. substitutions) that are introduced into a polypeptide. Alternatively, the nucleic acids may be changed from a wild-type sequence e.g. optimized for any of several reasons, such as to improve stability, to introduce or remove restriction sites, to accommodate vector insertion sites, to utilize residues that are plentiful or easily transcribed in a particular host species, etc. Such modifications, and others, are known in the art. The nucleic acids can be DNA, RNA, or hybrids thereof. Vectors comprising the nucleic acid sequences are also encompassed, many types of which are known in the art e.g. plasmids, viral vectors, yeast-based vectors, etc.

The nucleic acid sequences (DNA) that encode the Treponema denticola wild type DNA sequence of chimeras FhbB-ch5 and FhbB-ch4 codon-optimized versions thereof, are shown below.

1. The Treponema denticola wild type DNA sequence
of FhbB-ch5 chimera
(SEQ ID NO: 8)
ACCTTCAAAATGAATACCGCGCAGAAGGCCCATTATGAGAAGTTCATCAATGCCCTGGAGAACG
CCCTGAAAACCCGCCATATCCCTGCTGGTGCCGTTATCGACATGCTGGCCGAGATTAACACCGA
GGCCCIGGCACTGGACTATCAGATCGTGGATAAAAAACCGGGCACCAGCATTGCACAGGGTACC
AAGGCCGCCGCACTGCGTAAACGTTTTATTCCTAAGAAAATTAAAGCATTCAAGATGAATACCG
CACAGAAAGCACATTACGAAGCATTCATTAAAGTGCTGGAGAAGGCCGCCGAGCGCAACCCGAT
TGACGCACAGGTTGTTGTTGAAGCACTGGGCGCCGTTAACATCGACGCCCTGGCAAAAAACCTG
AACTATCAGGTGATTGACAAGAAGCCGGGCACCGATATTGCCACCGGTACCAAGGCCGCAGAGC
TGCGCAAGCGCTTCGTGCCGAAGAAAATTAAAGCCTTTAAAATGAACACCGCCCAGAAAGCCCA
TTACGAGGCATTCATCAGCGGTCTGGAAAATGCCGTGAAGGATAATCCGATGACCGCACAGAAC
GTTAAAGAAGGCCTGGACCTGGCAAATGTGGGCGCCGCAGCCCTGAACTTTAAGATTGTGGATA
AGAAAGCAGGTACCGAGATTGCCAAGGGCACCAAAGCCGCAGAACTGCGCAAACGCTTTGTGCC
GAAGAAAAAAGCCTTTAAGATGAATACCGCCCAGAACGCCCACTACGAAGCATTTATTAGCGGT
CTGGAGCGCGGTGCCAAAGATAACCCGATGCTGGCACAGGTTGTGAAGGCCGGCCTGGATCTGG
CCAATGATGGTGCCGCCGCACTGAACTACAAAATTGTGGATAAAAAGCCGGGCACCGACATCGC
CAAAGGTACCAAAGCCGCCGAACTGCGCAAACGTTTCATTCCGAAGAAAATTAAAACCTTCAAA
ATGAATACCGCCCAAAAGGCACACTATGAGGCATTCATTGCCGATCTGGAACGCGCCGCCAAGG
ACAATCCTATGCCGGCCCATATTGTGAAAGCAGGICTGGATGCCGCCAATGCAATCGCCGCCAC
CCTGAATTTCAAGATCGTGGACAAGAAGGCCGGCACAGAAATCGCCAAAGGCACCAAGGCCGCA
GAACTGCGCAAGCGCTTTGTGCCGAAAAAGAAA
2. The codon optimized (for Escherichia coli)
sequence of FhbB-ch5 chimera. Nucleotide
content: A 364 T 195 C 329 G 297|GC%:
52.83%|Length: 1185
(SEQ ID NO: 9)
ACATTTAAGATGAACACCGCCCAAAAGGCCCATTACGAGAAATTCATCAACGCCCTGGAAAACG
CCCTGAAGACCCGTCATATTCCTGCTGGTGCCGTGATTGATATGCTGGCCGAGATTAACACCGA
AGCCCTGGCCCTGGACTACCAGATCGTGGATAAGAAACCGGGCACCAGTATTGCCCAAGGTACC
AAGGCCGCCGCACTGCGCAAGCGCTTTATCCCGAAAAAGATTAAAGCCTTCAAGATGAACACCG
CCCAAAAAGCCCATTACGAGGCCTTCATTAAAGTGCTGGAAAAAGCCGCCGAGCGTAATCCGAT
CGATGCACAGGTGGTGGTTGAGGCCCTGGGCGCAGTGAATATTGACGCCCTGGCAAAAAATCTG
AACTACCAGGTGATCGACAAAAAGCCGGGCACCGACATTGCCACCGGTACCAAAGCAGCAGAAC
TGCGCAAACGCTTTGTGCCGAAGAAAATTAAGGCCTTCAAAATGAACACCGCCCAGAAAGCCCA
TTATGAGGCATTCATTAGCGGCCTGGAGAACGCCGTTAAAGACAACCCGATGACAGCCCAGAAC
GTGAAAGAAGGTCTGGACCTGGCCAATGTGGGCGCAGCAGCCCTGAATTTCAAAATTGTGGATA
AGAAGGCCGGCACCGAGATTGCCAAAGGCACCAAGGCCGCCGAGCTGCGCAAGCGCTTCGTGCC
GAAGAAAAAAGCCTTTAAGATGAATACCGCACAGAACGCCCATTACGAAGCCTTCATCAGCGGT
CTGGAACGTGGCGCAAAGGATAACCCGATGCTGGCCCAGGTTGTTAAAGCCGGTCTGGATCIGG
CAAACGATGGCGCCGCCGCACTGAACTATAAAATCGTGGACAAGAAGCCGGGTACCGATATTGC
CAAGGGCACCAAAGCAGCCGAACTGCGTAAACGCTTCATCCCGAAGAAGATTAAAACCTTTAAA
ATGAACACCGCACAAAAGGCCCACTACGAAGCCTTTATCGCCGATCTGGAACGTGCAGCCAAAG
ACAATCCGATGCCGGCCCACATTGTTAAGGCCGGTCTGGACGCAGCAAACGCCATCGCCGCCAC
CCTGAACTTTAAAATCGTGGACAAAAAGGCCGGTACCGAAATTGCCAAGGGCACCAAGGCCGCC
GAGCTGCGCAAACGTTTTGTTCCGAAAAAGAAA
3. Treponema denticola derived wild type
sequence for FhbB-ch4 chimera
(SEQ ID NO: 10)
ACCTTCAAAATGAATACCGCGCAGAAGGCCCATTATGAGAAGTTCATCAATGCCCTGGAGAACG
CCCTGAAAACCCGCCATATCCCTGCTGGTGCCGTTATCGACATGCTGGCCGAGATTAACACCGA
GGCCCTGGCACTGGACTATCAGATCGTGGATAAAAAACCGGGCACCAGCATTGCACAGGGTACC
AAGGCCGCCGCACTGCGTAAACGTTTTATTCCTAAGAAAATTAAAGCATTCAAGATGAATACCG
CACAGAAAGCACATTACGAAGCATTCATTAAAGTGCTGGAGAAGGCCGCCGAGCGCAACCCGAT
TGACGCACAGGTTGTTGTTGAAGCACTGGGCGCCGTTAACATCGACGCCCTGGCAAAAAACCTG
AACTATCAGGTGATTGACAAGAAGCCGGGCACCGATATTGCCACCGGTACCAAGGCCGCAGAGC
TGCGCAAGCGCTTCGTGCCGAAGAAAATTAAAGCCTTTAAAATGAACACCGCCCAGAAAGCCCA
TTACGAGGCATTCATCAGCGGTCTGGAAAATGCCGTGAAGGATAATCCGATGACCGCACAGAAC
GTTAAAGAAGGCCTGGACCTGGCAAATGTGGGCGCCGCAGCCCTGAACTTTAAGATTGTGGATA
AGAAAGCAGGTACCGAGATTGCCAAGGGCACCAAAGCCGCAGAACTGCGCAAACGCTTTGTGCC
GAAGAAAAAAGCCTTTAAGATGAATACCGCCCAGAACGCCCACTACGAAGCATTTATTAGCGGT
CTGGAGCGCGGTGCCAAAGATAACCCGATGCTGGCACAGGTTGTGAAGGCCGGCCTGGATCTGG
CCAATGATGGTGCCGCCGCACTGAACTACAAAATTGTGGATAAAAAGCCGGGCACCGACATCGC
CAAAGGTACCAAAGCCGCCGAACTGCGCAAACGTTTCATTCCGAAGAAAATTAAAACCTTCAAA
4. Codon optimized (for Escherichia coli)
coding sequence for FhbB-ch4 chimera
(SEQ ID NO: 11)
ACATTTAAGATGAACACCGCCCAAAAGGCCCATTACGAGAAATTCATCAACGCCCTGGAAAACG
CCCTGAAGACCCGTCATATTCCTGCTGGTGCCGTGATTGATATGCTGGCCGAGATTAACACCGA
AGCCCTGGCCCTGGACTACCAGATCGTGGATAAGAAACCGGGCACCAGTATTGCCCAAGGTACC
AAGGCCGCCGCACTGCGCAAGCGCTTTATCCCGAAAAAGATTAAAGCCTTCAAGATGAACACCG
CCCAAAAAGCCCATTACGAGGCCTTCATTAAAGTGCTGGAAAAAGCCGCCGAGCGTAATCCGAT
CGATGCACAGGTGGTGGTTGAGGCCCTGGGCGCAGTGAATATTGACGCCCTGGCAAAAAATCTG
AACTACCAGGTGATCGACAAAAAGCCGGGCACCGACATTGCCACCGGTACCAAAGCAGCAGAAC
TGCGCAAACGCTTTGTGCCGAAGAAAATTAAGGCCTTCAAAATGAACACCGCCCAGAAAGCCCA
TTATGAGGCATTCATTAGCGGCCTGGAGAACGCCGTTAAAGACAACCCGATGACAGCCCAGAAC
GTGAAAGAAGGTCTGGACCTGGCCAATGTGGGCGCAGCAGCCCTGAATTTCAAAATTGTGGATA
AGAAGGCCGGCACCGAGATTGCCAAAGGCACCAAGGCCGCCGAGCTGCGCAAGCGCTTCGTGCC
GAAGAAAAAAGCCTTTAAGATGAATACCGCACAGAACGCCCATTACGAAGCCTTCATCAGCGGT
CTGGAACGTGGCGCAAAGGATAACCCGATGCTGGCCCAGGTTGTTAAAGCCGGTCTGGATCTGG
CAAACGATGGCGCCGCCGCACTGAACTATAAAATCGTGGACAAGAAGCCGGGTACCGATATTGC
CAAGGGCACCAAAGCAGCCGAACTGCGTAAACGCTTCATCCCGAAGAAGATTAAAACCTTTAAA

Variants and modified versions of the sequences presented herein are also encompassed. Generally, variants of the polypeptides and nucleic acids disclosed herein have a high degree of identity or percent identity with the exemplary polypeptides and nucleic acids that are shown. The terms “identical” or percent “identity,” in the context of two or more polypeptide or nucleic acid sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site located at ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length. As used herein, percent (%) amino acid sequence identity is defined as the percentage of amino acids or nucleotides in a candidate sequence that are identical to the amino acids or nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

“Percentage of sequence identity” is determined by comparing two optimally aligned sequences over a comparison window, wherein the portion of the polypeptide or nucleotide sequence in the comparison window may comprise additions or deletions (i.e., gaps) as compared to the reference sequence for optimal alignment of the two sequences. The percentage is calculated by determining the number of positions at which the identical amino acid residue or nucleotide occurs in both sequences to yield the number of matched positions, dividing the number of matched positions by the total number of positions in the window of comparison and multiplying the result by 100 to yield the percentage of sequence identity.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of a number of contiguous positions selected from the group consisting of from 10 to 700, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)). The complete contents of each of these references is hereby incorporated by reference in entirety.

### Compositions

The compounds described herein are generally delivered (administered) as a pharmaceutical composition. The “compounds” refers to the chimeric proteins and/or antibodies directed against the chimeric proteins, i.e. compositions that are used as vaccines to elicit an immune response, or compositions comprising antibodies that are used e.g. for antibody therapy. Such pharmaceutical compositions generally comprise at least one of the disclosed chimeric proteins, i.e. one or more than one (a plurality) of different chimeras may be included in a single formulation; or a plurality of antibodies. Accordingly, the present invention encompasses such formulations/compositions. The compositions generally include one or more substantially purified chimeric proteins or antibodies as described herein, and a pharmacologically suitable (physiologically compatible) carrier, which may be aqueous or oil-based. In some aspects, such compositions are prepared as liquid solutions or suspensions, or as solid forms such as tablets, pills, powders and the like, or as semi-solid pastes, gels, etc. Solid forms suitable for solution in, or suspension in, liquids prior to administration are also contemplated (e.g. lyophilized forms of the compounds), as are emulsified preparations. For local oral delivery (especially for antibody preparations), the compositions may be formulated e.g. as a chewable gum, gel, paste (e.g. toothpaste), a rinse or mouth wash for direct delivery to the site of action (e.g. the gum of a subject and/or a periodontal pocket), and/or as a slow-release formulation as described below.

In some aspects, the active ingredients are mixed with excipients which are pharmaceutically acceptable and compatible with the active ingredients, e.g. pharmaceutically acceptable salts. Suitable excipients include, for example, water, saline, dextrose, glycerol, ethanol and the like, or combinations thereof. In addition, the compositions may contain minor amounts of auxiliary substances such as wetting or emulsifying agents, pH buffering agents, preservatives, and the like. If it is desired to administer an oral form of the composition, various thickeners, flavorings, diluents, emulsifiers, dispersing aids or binders and the like are added. The composition of the present invention may contain any such additional ingredients so as to provide the composition in a form suitable for administration. The final amount of compound in the formulations varies but is generally from about 1-99%. Still other suitable formulations for use in the present invention are found, for example in Remington's Pharmaceutical Sciences, 22nd ed. (2012; eds. Allen, Adejarem Desselle and Felton). The complete contents of this reference is hereby incorporated by reference in entirety.

Some examples of materials which can serve as pharmaceutically acceptable carriers include, but are not limited to, ion exchangers, alumina, aluminum stearate, lecithin, serum proteins (such as human serum albumin), buffer substances (such as Tween 80™, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

“Pharmaceutically acceptable salts” refers to the relatively non-toxic, inorganic and organic acid addition salts, and base addition salts, of compounds of the present invention. These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-β-hydroxynaphthoates, gentisates, isethionates, di-p-toluoyltartrates, methanesulfonates, ethanesulfonates, benzenesulfonates, p-toluenesulfonates, cyclohexylsulfamates and laurylsulfonate salts, and the like. See, for example S. M. Berge, et al., “Pharmaceutical Salts,” J. Pharm. Sci., 66, 1-19 (1977). The complete contents of this reference is hereby incorporated by reference in entirety. Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use. ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like.

The vaccine formulations may contain one or more adjuvants to potentiate the immune response to one or more antigens in the immunogenic composition. Suitable vaccine adjuvants for incorporation into the present formulation are described in the pertinent texts and literature and will be apparent to those of ordinary skill in the art. The major adjuvant groups are as follows: Mineral salt adjuvants, including alum-based adjuvants such as aluminum phosphate, aluminum hydroxide, and aluminum sulfate, as well as other mineral salt adjuvants such as the phosphate, hydroxide, and sulfate salts of calcium, iron, and zirconium; Saponin formulations, including the Quillaia saponin Quil A and the Quil A-derived saponin QS-21, as well as immune stimulating complexes (ISCOMs) formed upon admixture of cholesterol, phospholipid, and a saponin; Bacteria-derived and bacteria-related adjuvants, including, without limitation, cell wall peptidoglycans and lipopolysaccharides derived from Gram negative bacteria such as Mycobacterium spp., Corynebacterium parvum, C. granulosum, Bordetella pertussis, and Neisseria meningitis, such as Lipid A, monophosphoryl Lipid A (MPLA), other Lipid A derivatives and mimetics (e.g., RC529), enterobacterial lipopolysaccharide (“LPS”), TLR4 ligands, and trehalose dimycolate (“TDM”); Muramyl peptides such as N-acetyl muramyl-L-alanyl-D-isoglutamine (“MDP”) and MDP analogs and derivatives, e.g., threonyl-MDP and nor-MDP; Oil-based adjuvants, including oil-in-water (O/W) and water-in-oil (W/O) emulsions, such as squalene-water emulsions (e.g., MF59, AS03, AF03), complete Freund's adjuvant (“CFA”) and incomplete Freund's adjuvant (“IFA”); Liposome adjuvants; Microsphere adjuvants formed from biodegradable and non-toxic polymers such as a poly(a-hydroxy acid), a poly(hydroxy butyric) acid, a polyorthoester, a polyanhydride, a polycaprolactone, etc.; Human immunomodulators, including cytokines, such as interleukins (e.g. IL-1, IL-2, IL-4, IL-5, IL-6, IL-7, IL-12), interferons (e.g. interferon-gamma), macrophage colony stimulating factor, and tumor necrosis factor; Bioadhesives and mucoadhesives, such as chitosan and derivatives thereof and esterified hyaluronic acid and microspheres or mucoadhesives, such as cross-linked derivatives of poly(acrylic acid), polyvinyl alcohol, polyvinyl pyrrolidone, polysaccharides and carboxymethylcellulose; Imidazoquinolone compounds, including Imiquamod and homologues thereof, e.g., Resiquimod; TLR-9 agonists, such as Hsp90 and oligodeoxynucleotides containing unmethylated CpG motifs (see, e.g., Bode et al. (2011) Expert Rev. Vaccines 10(4): 499-511), the complete contents of which is hereby incorporated by reference in entirety; and Carbohydrate adjuvants, including the inulin-derived adjuvants gamma inulin and algammulin, and other carbohydrate adjuvants such as polysaccharides based on glucose and mannose, including glucans, dextrans, lentinans, glucomannans, galactomannans, levans, and xylans.

Exemplary adjuvants herein include alum-based salts such as aluminum phosphate and aluminum hydroxide.

In some aspects, particularly for the delivery of antibodies, the chimeric proteins are delivered via a “slow” or “controlled” or “extended” release delivery system, e.g. for local administration. Controlled release can be taken to mean any extended-release dosage forms. The following terms may be considered to be substantially equivalent to controlled release, for the purposes of the present disclosure: continuous release, controlled release, delayed release, depot, gradual release, long term release, programmed release, prolonged release, proportionate release, protracted release, repository, slow release, spaced release, sustained release, time coat, time release, delayed action, extended action, layered time action, long acting, prolonged action, sustained action, extended release, release in terms of pH level, etc.

Numerous controlled release vehicles are known, including biodegradable or bioerodable polymers such as polylactic acid, polyglycolic acid, and regenerated collagen. Known controlled release drug delivery devices include creams, lotions, tablets, capsules, gels, microspheres, liposomes, inserts, etc. Implantable or injectable polymer matrices, and transdermal and transmucosal formulations, from which active ingredients are slowly released, are also well known and can be used in the disclosed methods.

In some aspects, controlled release preparations are manufactured by and comprise, e.g. polymers to form complexes with or which absorb proteins. In some aspects, the controlled delivery is exercised by selecting appropriate macromolecules such as polyesters, polyamino acids, polyvinylpyrrolidone, ethylenevinyl acetate, methylcellulose, carboxymethylcellulose, protamine sulfate, etc., and the concentration of these macromolecule as well as the methods of incorporation are selected in order to control release of active complex. Other components of a slow-release formulation include but are not limited to: biodegradable pharmaceutically acceptable water-insoluble polymers in the form of a matrix; plasticizing agents; wetting agents, suspending and dispersing agents; enzymatically biodegradable pharmaceutically acceptable water soluble polymers, etc. Biodegradable water-insoluble polymers are degradable by enzymatic degradation, physical disintegration or a combination thereof.

Hydrogels, in which one or more active agents (e.g. chimeric proteins or antibodies) are dissolved in an aqueous constituent to gradually release over time, can be prepared by copolymerization of hydrophilic mono-olefinic monomers such as ethylene glycol methacrylate. Matrix devices, wherein one or more chimeric proteins are dispersed in a matrix of carrier material, can be used. The carrier matrix can be porous, non-porous, solid, semi-solid, permeable or impermeable. Alternatively, a device comprising a central reservoir of one or more chimeric proteins surrounded by a rate controlling membrane can be used to control the release. Rate controlling membranes include but are not limited to ethylene-vinyl acetate copolymer and butylene terephthalate/polytetramethylene ether terephthalate. Use of silicon rubber depots are also contemplated.

Additionally, with regard to the preparation of slow-release formulations, reference is made to U.S. Pat. Nos. 5,024,843, 5,091,190, 5,082,668, 4,612,008 and 4,327,725, the complete contents of each of which is hereby incorporated by reference herein. In addition, US published patent application 20120100192, the complete contents of which is hereby incorporated by reference in entirety, discloses an oral delivery composition for the treatment of periodontal disease which may be used for delivery of the proteins disclosed herein, the device being in a solid unit dosage form configured for insertion into a periodontal pocket of a patient.

Slow-release formulations may be especially applicable to the direct delivery of active agents, especially antibodies, to a site of action, such as the gum, periodontal pockets, etc. of a patient, or even the surrounding area, e.g. the teeth, tongue, sublingual area, roof of the mouth, cheek lining, etc.

The invention also provides pharmaceutical formulations that comprise the active agents in a sterile formulation for administration to a subject, e.g., as a suspension, solution or in lyophilized form to be rehydrated prior to use. After the compositions have been prepared, they can be placed in an appropriate container and labeled for treatment of an indicated condition. For administration of a composition of the invention, such labeling would include amount, frequency, and method of administration.

### Antibodies

Antibodies against the chimeric proteins disclosed herein are also provided. The term “antibody” includes polyclonal, monoclonal, or other purified preparations of antibodies, recombinant antibodies, monovalent antibodies, and multivalent antibodies. Antibodies may be humanized and may further include engineered complexes that comprise antibody-derived binding sites, such as diabodies and triabodies. The term “antibody” or “antibodies” may also refer to whole or fragmented antibodies in unpurified or partially purified form (e.g., hybridoma supernatant, ascites, polyclonal antisera) or in purified form. The antibodies may be of any suitable origin or form including, for example, murine (e.g., produced by murine hybridoma cells), or expressed as humanized antibodies, chimeric antibodies, human antibodies, and the like. For instance, antibodies may be wholly or partially derived from human (e.g., IgG (IgG1, IgG2, IgG2a, Ig2b, IgG3, IgG4), IgM, IgA (IgA1 and IgA2), IgD, and IgE), canine (e.g., IgGA, IgGB, IgGC, IgGD), chicken (e.g., IgA, IgD, IgE, IgG, IgM, IgY), goat (e.g., IgG), mouse (e.g., IgG, IgD, IgE, IgG, IgM), and/or pig (e.g., IgG, IgD, IgE, IgG, IgM), rat (e.g., IgG, IgD, IgE, IgG, IgM) antibodies, for instance. Methods of preparing, utilizing and storing various types of antibodies are well-known to those of skill in the art and would be suitable in practicing the present invention (see, for example, Harlow, et al. Antibodies: A Laboratory Manual, Cold Spring Harbor Laboratory, 1988; Harlow, et al. Using Antibodies: A Laboratory Manual, Portable Protocol No. 1, 1998; Kohler and Milstein, Nature, 256:495 (1975)); Jones et al. Nature, 321:522-525 (1986); Riechmann et al. Nature, 332:323-329 (1988); Presta (Curr. Op. Struct. Biol., 2:593-596 (1992); Verhoeyen et al. (Science, 239:1534-1536 (1988); Hoogenboom et al., J. Mol. Biol., 227:381 (1991); Marks et al., J. Mol. Biol., 222:581 (1991); Cole et al., Monoclonal Antibodies and Cancer Therapy, Alan R. Liss, p. 77 (1985); Boerner et al., J. Immunol., 147(1):86-95 (1991); Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995); as well as U.S. Pat. Nos. 4,816,567; 5,545,807; 5,545,806; 5,569,825; 5,625,126; 5,633,425; and, 5,661,016). The complete contents of each of these references is hereby incorporated by reference in entirety.

In certain applications, the antibodies may be contained within hybridoma supernatant or ascites and utilized either directly as such or following concentration using standard techniques. In other applications, the antibodies may be further purified using, for example, salt fractionation and ion exchange chromatography, or affinity chromatography using Protein A, Protein G, Protein A/G, and/or Protein L ligands covalently coupled to a solid support such as agarose beads, or combinations of these techniques. The antibodies may be stored in any suitable format, including as a frozen preparation (e.g., −20° C. or −70° C.), in lyophilized form, or under normal refrigeration conditions (e.g., 4° C.). When stored in liquid form, for instance, it is preferred that a suitable buffer such as Tris-buffered saline (TBS) or phosphate buffered saline (PBS) is utilized. In some embodiments, the binding agent may be prepared as an injectable preparation, such as in suspension in a non-toxic parenterally acceptable diluent or solvent. Suitable vehicles and solvents that may be utilized include water, Ringer's solution, and isotonic sodium chloride solution, TBS and/or PBS, among others. Such preparations may be suitable for use in vitro or in vivo may be prepared as is known in the art and the exact preparation may depend on the particular application.

As aspect of the invention provides isolated polyclonal antibodies. Those of skill in the art are familiar with techniques for producing and obtaining polyclonal antibodies. Generally, polyclonal antibodies (pAbs) are produced by injecting a specific antigen into lab animals (e.g. rabbits, goats, etc.). The animal is immunized repeatedly to obtain higher titers of antibodies specific for the antigen. Within a few weeks, polyclonal antibodies can be harvested and collected from the antiserum. Production of polyclonal antibodies is generally easier and more less expensive than the production of monoclonal antibodies. Furthermore, polyclonal antisera can be generated in a shorter time (4-8 weeks), whereas it takes about 3 to 6 months to produce mAbs.

An aspect of the invention provides isolated monoclonal antibodies (e.g., recombinant humanized, chimeric, and human antibodies) which exhibit therapeutically advantageous patterns of binding to FhbB protein. The term “monoclonal antibody,” as used herein, refers to an antibody that displays a single binding specificity and affinity for a particular epitope or a composition of antibodies in which all antibodies display a single binding specificity and affinity for a particular epitope. For example, the monoclonal antibodies to be used in accordance with the present invention may be made by the hybridoma method first described by Kohler et al., (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al., (1991) Nature 352: 624-628 and Marks et al., (1991) J. Mol. Biol. 222: 581-597.

In some aspects, the monoclonal antibodies are produced by injecting a chimeric protein as described herein is injected into a host animal, such as a mouse. The host animal naturally produces lymphocytes, which produce antibodies specific to the antigen, and spleen cells which produce the lymphocytes are removed from the host. The spleen cells are fused with human cancerous white blood cells called myeloma cells to form hybridoma cells which divide indefinitely and produce while producing monoclonal antibodies.

Antigen binding fragments (including scFvs) of such immunoglobulins are also encompassed by the term “monoclonal antibody” as used herein. Monoclonal antibodies are highly specific, being directed against a single antigenic site. Furthermore, in contrast to conventional (polyclonal) antibody preparations, which typically include different antibodies directed against different epitopes on the antigen, each monoclonal antibody is directed against a single epitope. Monoclonal antibodies can be prepared using any art recognized technique and those described herein such as, for example, a hybridoma method, a transgenic animal, recombinant DNA methods (see, e.g., U.S. Pat. No. 4,816,567), or using phage antibody libraries using the techniques described in, for example, U.S. Pat. No. 7,388,088 and PCT Pub. No. WO 00/31246). Monoclonal antibodies include chimeric antibodies, human antibodies, and humanized antibodies and may occur naturally or be produced recombinantly.

The monoclonal antibodies herein also include camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079, which are hereby incorporated by reference in their entireties). In one embodiment, the present invention provides single domain antibodies comprising two V_{H} domains with modifications such that single domain antibodies are formed.

Immunoconjugates can be made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). Carbon-14-labeled 1-isothiocyanatobenzyl-3-methyldiethylene triaminepentaacetic acid (MX-DTPA) is an exemplary chelating agent for conjugation of radionucleotide to the antibody (see, e.g., PCT publication number WO94/11026).

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

### Administration

Also encompassed herein are methods of administering the agents described herein to treat and/or prevent PD. The term “treating” refers to therapeutic treatment by the administration of an immunogenic composition or vaccine formulation of the invention, where the object is to lessen or eliminate infection that already exists. For example, “treating” may include directly affecting, suppressing, inhibiting, and eliminating infection (for example, when a vaccine is protective), as well as reducing the severity of, delaying the onset of, and/or reducing symptoms associated with an infection. For example, as used herein, in some aspects, the term treating may include reducing the population of T. denticola present in the oral cavity of a subject, e.g. at the gum and/or in periodontal pockets.

“Preventing” (or prophylaxis or prophylactic treatment) generally refers, for example, to reducing the risk that a subject will develop one or more symptoms of an infection, delaying the onset of symptoms, preventing relapse of an infection, or preventing the development of infection, especially in a subject that is at risk of an infection. Administration of the compositions disclosed herein may both treat existing infections and prevent the future occurrence or re-occurrence of an infection. In order to delay the onset of the one or more of the underlying symptoms related to PD, the prevention, treatment and/or amelioration of symptoms need not be complete, so long as at least one symptom of the disease is prevented, treated and/or ameliorated. Typical symptoms of PD include but are not limited to: inflammation (e.g. gum inflammation), tooth abcesses, bad breath, red and swollen gums, tender or bleeding gums, painful chewing, loose and sensitive teeth, receding gums, longer appearing teeth, etc.

In some aspects, the methods involve administering to a subject in need thereof a therapeutically effective amount (e.g. an immunologically effective amount) of a periodontal formulation comprising the chimeric proteins described herein. Such a subject may be suffering from periodontitis or may be at risk of developing periodontitis. When the vaccine is used prophylactically, the subject may be predisposed to developing periodontitis as a result of any number of risk factors, including age; a genetic predisposition; an immunocompromised state; a disease that increases the risk of developing moderate to severe periodontitis, such as diabetes mellitus, AIDS, leukemia, Down's syndrome; or the presence of endodontic lesions or abscesses. As an example, patients receiving anti-TNF therapy (i.e., taking a TNF inhibitor such as etanercept or adalimumab), such as in the treatment of rheumatoid arthritis or psoriasis, often exhibit gingival inflammation and have an elevated risk of developing periodontitis.

The subject is generally a mammal, and typically a human. However, the treatment of non-human mammals is also encompassed, as long as non-human mammal harbors T. denticola FhbB and can benefit from the methods disclosed herein. Examples of e.g. veterinary subjects include but are not limited to: dogs, horses, dairy cattle, cats, apes, or other mammals. A “therapeutically effective amount” or an “immunologically effective amount” of the vaccine formulation is an amount that, either as a single dose or as part of a series of two or more doses, is effective for treating or preventing periodontal disease. The amount administered will vary according to several factors, including the overall health and physical condition of the subject, the subject's age, the capacity of the subject's immune system to synthesize relevant antibodies, the form of the composition (e.g., injectable liquid, nasal spray, etc.), the taxonomic group of the subject (e.g., human, non-human primate, non-primates, etc.), and other factors known to the medical practitioner overseeing administration. Generally, the amount ranges from about 1-1000 ug of chimera per dose, such as about 5 to 500 ug per dose, or more usually about 10-100 ug per dose, e.g. about 10, 20, 30, 40, 50, 60, 70, 80, 90 or 100 ug of chimeric protein per dose of vaccine composition.

In other aspects, the methods involve administering to a subject in need thereof a therapeutically effective amount of a periodontal formulation comprising antibodies against one or more antigens of the chimeric proteins described herein, i.e. the method is a method of antibody therapy. Generally, the amount of antibody ranges from about 1-1000 ug per dose, such as about 5 to 500 ug per dose, or more usually about 10-100 ug per dose, e.g. about 10, 20, 30, 40, 50, 60, 70, 80, 90 or 100 ug of antibody per dose of the composition. The production of suitable antibodies is discussed elsewhere herein.

The compositions disclosed herein are administered in vivo by any suitable route adapted to the goal of administration. For a vaccine, administration routes include but are not limited to: inoculation or injection (e.g. intravenous, intraperitoneal, intramuscular, subcutaneous, intraarticular, and the like), and by absorption through epithelial or mucocutaneous linings (e.g., nasal, oral, and the like). Other suitable means include but are not limited to: inhalation (e.g. as a mist or spray), orally (e.g. as a pill, capsule, liquid, etc.), etc. A vaccine composition is administered systemically.

In some aspects, especially for the administration of antibodies, the mode of administration is local, such as directly to the gums or oral cavity of a patient. Such administration may be topical, by injection into the gums and/or by a slow-release composition placed e.g. directly into a periodontal pocket (a periodontal implant). Further, the antibodies may be delivered locally by being incorporated into dressings or bandages (e.g. lyophilized forms may be included directly in the dressing) which are placed in contact with the gums.

The compositions may be self-administered or administered by a medical professional. If self-administered, the compositions are generally in the form of e.g. a paste, gel, or spray or embedded in dental floss for local delivery. These forms may also be used by a professional, but a professional may also deliver the compositions e.g. by more invasive means, e.g. injection, implants, etc.

In addition, the compositions may be administered in conjunction with other treatment modalities such as substances that boost the immune system, various chemotherapeutic agents, various antibiotic agents, various anti-inflammatory agents, agents that act to kill or inhibit other oral pathogens that are involved in PD, and the like. Examples of other oral pathogens that may be targeted in treatments that are administered together with or in coordination with the present treatments include but are not limited to: Porphyromonas gingivalis (e.g. as described in published US patent application 20190192645, the entire contents of which is herein incorporated by reference in entirety, which describes targeting the Mfa1 fimbrilin protein of a Porphyromonas bacterium); Tannerella forsythia; the bacterial isolates described in published US patent applications 20080311151, the entire contents of which is herein incorporated by reference in entirety, Fusobacterium species, T. maltophilum, T socranskii, T. vincentii, T. pectinovorum, T. putida, and other oral treopnemes, etc.

The vaccine or immunogenic compositions are administered to a subject within the context of an appropriate dosage regimen. The composition may be administered once, or two or more times spaced out over an extended time period. For example, an initial, “prime” dose may be followed by at least one “boost” dose. The time interval between the prime and the subsequent boost dose, and between boost doses, is usually in the range of about 2 to about 24 weeks, more typically in the range of about 2 to 12 weeks, such as 2 to 8 weeks, 3-6 weeks, etc. Regardless of the mode of administration, e.g., intramuscular injection, gingival injection, intranasal administration, or the like, the volume of a single dose of the vaccine will generally be in the range of about 1 μL to about 500 μL, typically in the range of about 1 μL to about 250 μL, more typically in the range of about 2.5 μL to about 200 μL, and preferably in the range of about 5 μL to about 150 μL. It will be appreciated that the concentration of total antigen in the immunogenic composition corresponds to an immunologically effective dose of the composition per unit volume, working from these dose volume guidelines. Suggested amounts are described elsewhere herein.

For ease of use, a vaccine or immunogenic composition of the invention can be incorporated into a packaged product, or “kit,” including instructions for self-administration or administration by a medical practitioner. The kit includes a sealed container housing a dose of the vaccine formulation, typically a “unit dose” appropriate for a single dosage event that is immunologically effective. The vaccine may be in liquid form and thus ready to administer as an injection or the like, or it may be in another form that requires the user to perform a preparation process prior to administration, e.g., hydration of a lyophilized formulation, activation of an inert component, or the like. The kit may also include two or more sealed containers with the prime dose in a first container and a boost dose in one or more additional containers, or a periodontitis vaccine formulation in a first container and a vaccine directed against another infection in another container.

Antibody formulations for local delivery may also be packaged into a kit comprising e.g. a individual doses in the form of gums, hydrogels or other slow release compositions, or as a liquid wash, etc. For such purposes, sterile blister packs may be used. The frequency of local administration generally ranges from about 1-4 times per day, week or month and may depend on the severity of disease. Follow-up doses may be administered e.g. at monthly intervals after 1-4 weeks of intense, daily or bi-daily treatment. Any treatment regimen that results in treatment and/or prevention of PD may be employed

### Additional Methods

In some aspects, what is disclosed is a method of producing (generating, eliciting, etc.) polyclonal antibodies to FhbB, the method comprising inoculating a host animal with at least one chimera as described herein under conditions and for a period of time that permits the host animal to generate antibodies to the chimera(s); and then harvesting the polyclonal antibodies. Such polyclonal antibodies may be used in methods of preventing and treating PD, e.g. generally by local, direct application of the antibodies to a site of infection, as described above. Polyclonal antibodies made by this process are also encompassed.

In preferred aspects, monoclonal antibodies are produced by injecting a chimera into a subject (e.g. a laboratory animal) to generate spleen cells that produce lymphocytes that secrete a single type of antibody i.e. a monoclonal antibody to one antigen of the chimera. The spleen cells are harvested and rendered immortal by fusion to an immortal cell line. Monoclonal antibodies produced in this manner are harvested and used to treat and/or prevent PD, e.g. by local administration as described herein. Monoclonal antibodies made by this process are also encompassed.

Also provided are methods of blocking FH cleavage by FhbB. The methods may be performed in vitro or in vivo. The methods involve contacting the FH with anti-FhbB antibodies in an amount and under conditions sufficient to block FH cleavage. The antibodies may be present in antisera or may have been harvested from antisera (polyclonal antibodies) or from hybridoma cells cultured in vitro (monoclonal antibodies) as described elsewhere herein.

Methods of reducing the population of T. denticola present in the oral cavity of a subject, e.g. at the gum and/or in periodontal pockets, are also provided. The methods comprise i) administering to the subject a therapeutically effective amount of a composition comprising the chimeric proteins disclosed herein, or ii) administering to the subject a therapeutically effective amount of antibodies to the chimeric proteins. In the latter case, the antibodies may be polyclonal but are preferably monoclonal, and are administered locally, e.g. using a sustained release formulation.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### Example

Treponema denticola is a proteolytic anaerobic spirochete and key contributor to periodontal disease of microbial etiology. As periodontal disease develops and progresses, T. denticola thrives in the hostile environment of the subgingival crevice by exploiting the negative regulatory activity of the complement protein, factor H (FH). FH bound to the cell surface receptor, FhbB (FH binding protein B), is competent to serve as a cofactor for the Factor I mediated-cleavage of the opsonin C3b. However, bound FH is ultimately cleaved by the T. denticola protease, dentilisin. As the T. denticola population expands, the rate of FH cleavage may exceed its rate of replenishment leading to local FH depletion and immune dysregulation culminating in tissue and ligament destruction and tooth loss.

This example describes the development of an exemplary T. denticola FhbB based-vaccine antigen that blocks FH binding and cleavage and kill T. denticola cells via antibody-mediated bactericidal activity.

Tetra (FhbB-ch4) and pentavalent fhbB (FhbB-ch5) chimerics were engineered to have attenuated FH binding ability. The chimerics were immunogenic and elicited high-titer bactericidal and agglutinating antibody. Anti-Fhb-ch4 antisera blocked FH binding and cleavage by the T. denticola protease, dentilisin, in a dose dependent manner. This work is the first to take this approach to the development of a preventive or therapeutic vaccine (or monoclonal Ab) for periodontal disease.

### Materials and Methods

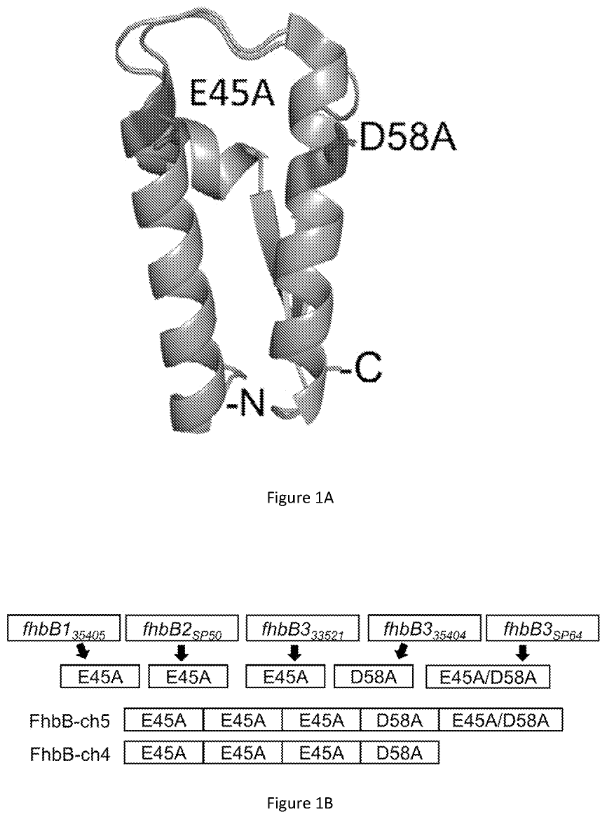

Bacterial strain cultivation, FhbB type identity, and growth conditions. All T. denticola strains including 35405 (FhbB1), SP50 (FhbB2), 33521 (FhbB3), 35404 (FhbB3), and SP64 (FhbB3) were cultivated in New Oral Spirochete (NOS) medium under anaerobic conditions (5% H₂; 20% CO₂; 75% N2; 37° C.). Cell growth was monitored using wet mounts and dark-field microscopy.
Site-directed mutagenesis, gene synthesis and generation of recombinant proteins.
Wild-type fhbB genes were PCR amplified from T. denticola strains 35405, 35404, SP50, SP64 and 33521 using primers with ligase independent cloning (LIC) tails and Phusion polymerase as recommended by the supplier (New England Biolabs). Signal peptide encoding sequences were omitted from each gene to enhance expression in Escherichia coli. The amplicons were prepared for LIC, annealed with linearized pET46-Ek LIC vector (Novagen), and the resulting plasmids propagated in E. coli NovaBlue cells (Novagen) as previously described (Miller et al., 2016). Plasmids were purified using QIAquik PCR Purification kits (Qiagen) and transformed into E. coli BL21 (DE3) cells. Protein production was induced with 1 mM Isopropyl β-d-1-thiogalactopyranoside (IPTG) and the proteins subsequently purified from the soluble fraction using nickel affinity chromatography and an AKTA Fast Protein Liquid Chromatography (FPLC) (GE Healthcare). All recombinant proteins were produced with an N-terminal hexa-histidine tag. Gene sequences were verified on a fee for service basis (Genewiz). Genes encoding FhbB proteins with single or double site-directed amino acid mutations were designed based on earlier studies (Miller et al., 2012; Miller et al., 2013; Tegels et al., 2018). The genes were codon optimized, synthesized and provided by the supplier in pUC57 (Genscript). The fhbB genes were PCR amplified from pUC57 with LIC primers and annealed with linearized pET46-Ek LIC. The plasmids were propagated in E. coli NovaBlue cells, expressed by IPTG induction in E. coli BL21 (DE3) cells, and purified as indicated above. fhbB chimerics (FhbB-ch4 and FhbB-ch5) consisting of the mutated fhbB genes (FIG. 1) were synthesized, cloned and protein production induced with IPTG as detailed above. All gene synthesis and cloning methods were as previously described (Miller et al., 2016). Note that subscripts are used throughout to indicate the isolate of origin of a given FhbB protein as needed (e.g., FhbB1₃₅₄₀₅).
Generation of antisera. Antisera were generated in Sprague-Dawley rats as previously described (Izac et al., 2020). In brief, rats were anesthetized with isoflurane, injected intraperitoneally with 40 μg of each recombinant protein in Freund's Complete adjuvant (Day 0) and then boosted with 40 μg of protein in Freund's Incomplete adjuvant (Days 21 and 35). On Day 42 the rats were euthanized, blood was collected by cardiac puncture, and serum harvested using standard methods. All animal experiments were conducted following the Guide for the Care and Use of Laboratory Animals (eighth edition) and in accordance with protocols peer-reviewed and approved by Virginia Commonwealth University Institutional Animal Care and Use Committees.
ELISA analyses. ELISAs were conducted as previously described (Izac et al., 2020). In brief, ELISA plate wells (in triplicate) were coated with protein (1 μg per well; bicarbonate buffer; overnight; 4° C.). All blocking, washing steps and Ab addition steps were with done with 5% non-fat milk (Carnation) in phosphate buffered saline with 0.5% Tween®20 (PBST). Antisera or preimmune sera (as indicated in the figures) was added (1:100) and incubated for 2 hr at room temperature. After washing, horseradish peroxidase (HRP) conjugated goat anti-rat IgG was added (1:15000; Pierce). The plates were washed and IgG binding was determined by measuring absorbance at 405 nm (Biotek Elx-808 μlate reader; Biotek). IgG titers were determined using the corresponding recombinant protein as the immobilized antigen (500 ng per well). Three-fold serial dilutions of sera ranging from 1:50 to 1:109350 were added. IgG binding was measured as above and log-transformed titers calculated at ⅓ OD max.
FH binding assays. FH binding to recombinant proteins was assessed using an ELISA format as detailed above. After immobilization of each protein, 5% non-fat milk in PBST was added and the plates were washed. Human FH was added (CompTech; 10 μg mL⁻¹ in PBST; 1 hr), the plates were washed and goat-anti human FH (1:1000) was added. IgG binding was detected using HRP-conjugated rabbit anti-goat IgG (1:20000; Pierce). Absorbance was measured as above.

### Indirect Immunofluorescence Assay (IFA).

Cells from mid-log phase cultures were air-dried onto glass slides. Non-specific Ab binding was blocked using PBST-B (PBST; 3% bovine serum albumin). Slides were screened with the appropriate antisera or preimmune sera (1:100; data not shown). Coverslips were mounted (ProLong Gold; Molecular Probes) and Alexa Fluor 568-conjugated goat anti-rat IgG added (1:1000 Molecular Probes). Cells were visualized by dark field microscopy and by fluorescence microscopy (BX51; Olympus).
Cell aggregation assays. To determine if anti-FhbB-ch4 antisera has bactericidal activity or can cause cell aggregation, mid-log phase T. denticola cultures (20 μl aliquots) were mixed with 40 μl of NOS media, 20 μl of heat inactivated (HI) anti-FhbB ch4 antisera sera and 20 μl of complement preserved Guinea Pig Serum (GPS; CompTech) or GPS (56° C.; 30 min). As a control, cells were incubated with GPS in the absence of antibody. The samples were transferred to glass slides and assessed for cell aggregation, membrane disruption and diminished motility at 15-minute intervals using dark-field microscopy. Note that percent killing could not be numerically expressed due to cell destruction and strong aggregation upon exposure to antisera in the presence of GPS.

### FH Cleavage Assays.

T. denticola 35405 (dentilisin positive phenotype) and SP50 (dentilisin deficient phenotype) cells (Miller et al., 2014) (0.1 OD₆₀₀ unit) were suspended in 50 μl of PBS containing purified human FH (40 μg mL⁻¹; Complement Tech). Antisera was added to achieve final concentrations of 0, 0.5, 1.0, and 10% (vol/vol). Samples were incubated for 0 or 60 min at 37° C., aliquots were removed for SDS-PAGE and immunoblotting. FH was detected using goat anti-human FH antisera (1:1000; CompTech) and IgG binding detected using HRP-conjugated rabbit anti-goat IgG (1:40000; Calbiochem).

### Results and Discussion

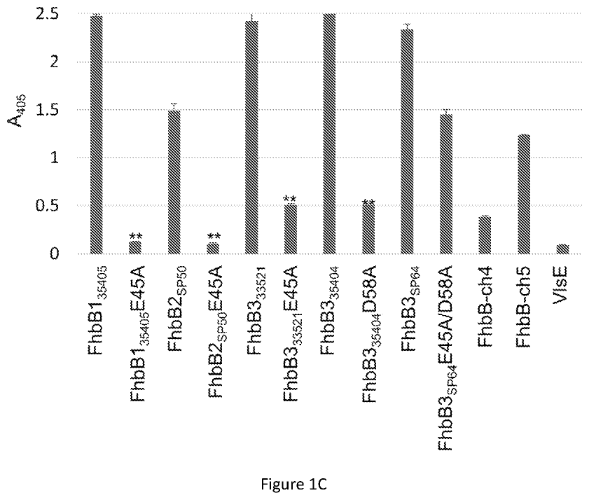

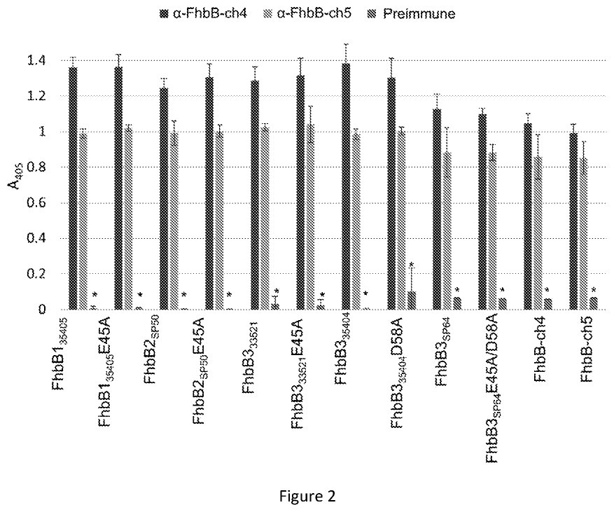

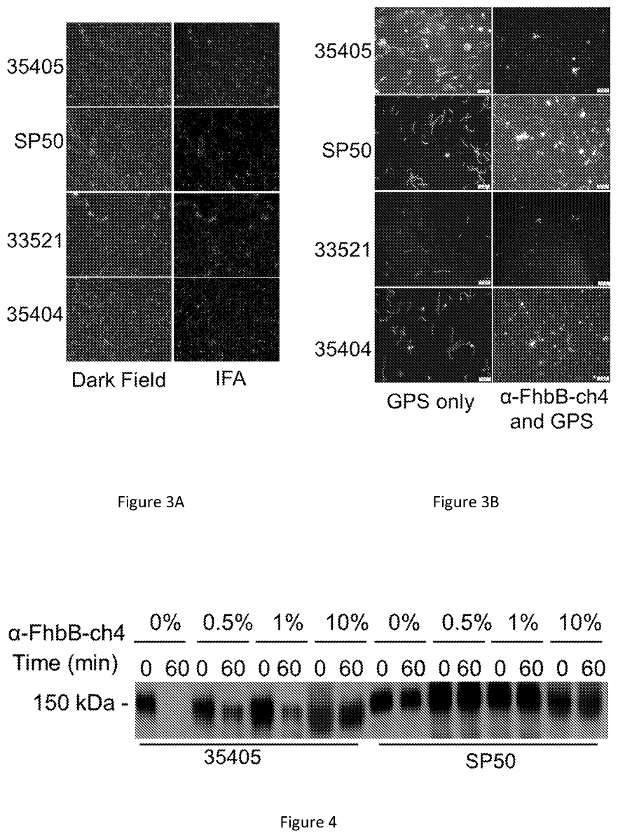

Production of Recombinant FhbB Chimerics with Attenuated FH Binding Ability.
Recombinant wild-type FhbB1, FhbB2, and FhbB3 (and divergent FhbB3 variants) with single or double-amino acid substitutions were successfully produced by E. coli as soluble proteins. The rationale for introducing site-directed amino acid substitutions into the FhbB proteins was to prevent FH binding to the protein upon administration to rats and thereby expose the FH binding interface for antibody generation and recognition. The atomic structure of FhbB has the side chains of residues E45 and D58 projecting outward from the negatively charged FH binding interface (FIG. 1). We previously demonstrated that alanine substitution of one or both of these residues of FhbB1 abolishes FH binding (Miller et al., 2012). To determine if the substitutions introduced into FhbB2 and FhbB3 variants also abolish FH binding, ELISA based-binding assays were conducted. Wild-type FhbB1₃₅₄₀₅, FhbB2_{SP50}, FhbB3₃₃₅₂₁, FhbB₃₃₅₄₀₄ and FhbB3_{SP64} bound human FH whereas the E45A or D58A site-directed mutants did not (FIG. 1). FH binding to FhbB3_{SP64}E45A/D58A was attenuated but not completely eliminated. Recombinant VlsE from Borreliella burgdorferi served as a negative control for FH binding and as expected, binding was not observed.
With the demonstration that FH binding was attenuated or abolished with the mutated proteins, the individual fhbB gene sequences (minus the segments encoding the leader peptides) were used to generate fhbB tetravalent (fhbB-ch4) and pentavalent (fhbB-ch5) chimeric constructs (FIG. 1). Both chimerics were readily expressed in E. coli as soluble proteins and purified cleanly using Ni-affinity chromatography. ELISA analyses of sera collected from immunized rats revealed that the chimerics are immunogenic and elicit high-titer IgG responses in rats (log transformed titers ranging from 4.0-4.9; data not shown). While FhbB-ch4 did not bind FH, residual binding to FhbB-ch5 was observed (FIG. 1). Single-dilution ELISA analyses verified that the individual FhbB variants represented in each chimeric are recognized by the anti-FhbB-ch4 and anti-FhbB-ch5 antisera (FIG. 2). Importantly, anti-FhbB-ch4 antisera recognized the FhbB_{SP64}E45A/D58A protein which is not directly represented in the FhbB-ch4 antisera. This suggests that antisera to the polyvalent chimeric can recognized diverse FhbB variants. In light of, and since FhbB-ch5 retained residual FH binding ability, subsequent analyses were primarily focused on the FhbB-ch4 chimeric vaccinogen.
Antisera to the FhbB chimerics binds to diverse T. denticola strains and causes cell aggregation and lysis. To determine if vaccinal antibody recognizes FhbB epitopes in the context of the bacterial cell membrane, IFA analyses were performed using non-permeabilized cells. The antibody elicited by immunization with anti-FhbB ch4 bound to strains producing divergent FhbB proteins (FIG. 3; Panel A). To determine if anti-FhbB-ch4 antisera is bactericidal or can trigger agglutination, strains expressing each FhbB type were incubated with HI-anti-FhbB-ch4 antisera and GPS or with GPS alone. Cells incubated with HI anti-FhbB-ch4 antisera plus GPS displayed significant membrane disruption, lack of motility, cell lysis and or aggregation (FIG. 3; Panel B) whereas cells incubated with GPS alone were unaffected demonstrating that killing and aggregation occurs through an antibody-dependent mechanism.
Anti-FhbB-ch4 antisera blocks FH binding and cleavage.
If vaccinal antibody can compete with FH for binding to FhbB then it would be expected to inhibit FH cleavage by dentilisin in a dose-dependent manner. Precedent for this was established in an earlier study that focused on individual FhbB variants (Miller et al., 2016). In the absence of anti-FhbB-ch4 antisera, all input FH was degraded by strain 35405 but not by strain SP50 which lacks dentilisin activity (FIG. 4). It can be concluded that anti-FhbB-ch4 antisera effectively blocks FH cleavage.

### CONCLUSIONS

It is somewhat of a paradox that while T. denticola survival in serum is dependent on FH binding, FH bound to the cell surface is cleaved and inactivated by the protease, dentilisin. Local FH depletion in periodontal pockets would lead to increased production of pro-inflammatory cytokines, accumulation and deposition of C3b on cell surfaces, and general immune dysregulation. Collectively, these outcomes would serve to drive the progression and development of PD. Consistent with its disproportionate contribution to PD, relative to other periopathogens, T. denticola is considered to be a keystone pathogen. The data presented above demonstrate that FhbB chimeric vaccinogens can be employed as preventative or therapeutic vaccines for PD.

While the invention has been described in terms of its several exemplary embodiments, those skilled in the art will recognize that the invention can be practiced with modification within the spirit and scope of the appended claims. Accordingly, the present invention should not be limited to the embodiments as described above but should further include all modifications and equivalents thereof within the spirit and scope of the description provided herein.

1. A recombinant chimeric protein comprising at least one genetically engineered mutant Treponema denticola Factor H Binding Protein B (FhbB) which comprises at least one mutation compared to a wild type FhbB primary sequence, wherein the at least one mutation prevents binding of the genetically engineered mutant T. denticola FhbB to Factor H (FH).

2. The recombinant chimeric protein of claim 1, wherein the at least one mutation includes a substitution at amino acid position 42, 43, 45, 57, 58, 64, 68, 93 and/or 96 of wild type FhbB primary sequence.

3. The recombinant chimeric protein of claim 1, wherein the at least one mutation is at one or both of amino acid positions 45 and 58.

4. The recombinant chimeric protein of claim 1, wherein the at least one mutation is an alanine substitution.

5. The recombinant chimeric protein of claim 1, wherein the recombinant chimeric protein comprises a plurality of genetically engineered mutant T. denticola FhbBs.

6. The recombinant chimeric protein of claim 1, wherein the recombinant chimeric protein comprises 2, 3, 4, 5 or 6 genetically engineered mutant T. denticola FhbBs.

7. The recombinant chimeric protein of claim 1, wherein the at least one genetically engineered mutant T. denticola FhbB has an amino acid sequence as set forth in SEQ ID NO: 1, SEQ ID NO: 2, SEQ ID NO: 3, SEQ ID NO: 4 or SEQ ID NO: 5.

8. The recombinant chimeric protein of claim 1, having an amino acid sequence as set forth in: SEQ ID NO: 6 or SEQ ID NO: 7.

9. A vaccine composition, comprising
a recombinant chimeric protein of claim 1.

10. A method of preventing and/or treating periodontal disease in a subject in need thereof, comprising,
administering to the subject
i) a therapeutically effective amount of the recombinant chimeric protein of claim 1; and/or
ii) a therapeutically effective amount of antibodies against the recombinant chimeric protein of claim 1.

11. The method of claim 10, wherein the therapeutically effective amount of the recombinant chimeric protein is administered systemically.

12. The method of claim 10, wherein the antibodies are monoclonal antibodies.

13. The method of claim 10, wherein the therapeutically effective amount of antibodies is administered locally.

14. The method of claim 13, wherein the therapeutically effective amount of antibodies is administered locally using a sustained-release formulation.

15. A method of eliciting an immune response to Treponema denticola Factor H Binding Protein B (FhbB) protein in a subject, comprising
administering to the subject an amount of a recombinant chimeric protein of claim 1, wherein the amount is sufficient to elicit an immune response in the subject.

16. The method of claim 15, wherein the immune response results in a reduction in the population of T. denticola in the subject.

17. The method of claim 15 wherein the immune response includes the production of antibodies.

18. The method of claim 15, further comprising harvesting the antibodies from the subject.

19. A method of producing monoclonal antibodies to the chimeric protein of claim 1, comprising
injecting the chimeric protein of claim 1 into a host animal;
obtaining spleen cells from the host animal;
fusing the spleen cells with myeloma cells to form hybridoma cells; and
culturing the hybridoma cells under conditions that permit lymphocytes within the hybridoma cells to produce the monoclonal antibodies.

20. A monoclonal antibody produced by the method of claim 19.
